(19) United States
(12) Patent Application Publication
(10) Pub. No.: US 2014/0330256 A1
(43) Pub. Date: Nov. 6, 2014

(54) Implantable Device for Manipulating Immune Cells

(71) Applicant: Elwha LLC, Bellevue, WA (US)

(72) Inventors: Roderick A. Hyde, Redmond, WA (US); Lowell L. Wood, JR., Bellevue, WA (US)

(21) Appl. No.: 13/875,409

(22) Filed: May 2, 2013

(51) Int. Cl. A61M 5/172

(52) U.S. Cl. CPC A61M 5/1723
USPC 604/891.1

(57) ABSTRACT

Systems, devices, methods, and compositions are disclosed which include an actively-controllable implantable device configured to, for example, capture and/or release biochemical and/or biological cells in a subject.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority application(s)). In addition, the present application is related to the “Related Applications,” if any, listed below.

### PRIORITY APPLICATIONS

None.

### RELATED APPLICATIONS

U.S. patent application Ser. No. To be Assigned, entitled IMPLANTABLE DEVICE FOR MANIPULATING IMMUNE CELLS, naming Roderick A. Hyde and Lowell L. Wood, Jr. as inventors, filed 2 May 2013 with attorney docket no. 0912-002-007-000000, is related to the present application.
U.S. patent application Ser. No. To be Assigned, entitled IMPLANTABLE DEVICE FOR MANIPULATING IMMUNE CELLS, naming Roderick A. Hyde and Lowell L. Wood, Jr. as inventors, filed 2 May 2013 with attorney docket no. 0912-002-008-000000, is related to the present application.

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

Described herein include various embodiments related to devices, systems, and methods for stimulating immune cells, and in particular for activating antigen presenting cells, optionally in a biological subject. In an embodiment, an implantable device, comprises a body structure having a surface including one or more sensors; and one or more actuatable reservoirs configured to direct an emitted biochemical or biological cell to one or more regions proximate the surface of the body structure and to deliver a patterned immune cell stimulus to the one or more regions proximate the surface of the body structure.

In an embodiment, an implantable system (such as a fluid management system) comprises a device (e.g., a microfluidics device) having a body structure including a surface with one or more sensors; and a plurality of independently actuatable immune cell stimulus delivering reservoirs configured to deliver a patterned immune cell stimulus to the one or more regions proximate the surface of the body structure, the plurality of independently actuatable immune cell stimulus delivering reservoirs defining at least a portion of the surface of the body structure. In an embodiment, a method of regulating an immune cell response from an at least partially implanted microfluidic device comprises selectively and actively releasing one or more biochemicals or one or more biological cells to one or more regions proximate the surface of an implanted portion of the microfluidic device via one or more actuatable reservoirs, and delivering a patterned immune cell stimulus composition to the one or more regions proximate the surface of the implanted portion of the microfluidic device in response to an automatically detected parameter associated with a biological sample proximate the surface of the implanted portion of the microfluidic device via one or more sensors of the device. In an embodiment, the independently actuatable immune cell stimulus delivering reservoirs are assigned to emit a first immune cell stimulus, and one or more of the plurality of independently actuatable reservoirs are assigned to emit a second immune cell stimulus. In an embodiment, the first immune cell stimulus and the second immune cell stimulus are the same stimulus. In an embodiment, the first immune cell stimulus and the second immune cell stimulus are different stimuli.

In an embodiment, the first immune cell stimulus or second immune cell stimulus includes at least one of vaccination, prophylactic treatment, or responsive treatment for disease. In an embodiment, the vaccination, prophylactic treatment, or responsive treatment for disease relate to different diseases for the first immune cell stimulus compared to the second immune cell stimulus. In an embodiment, multiple different independent actuatable reservoirs of the plurality each correspond to at least one different immune cell stimulus from the others. In an embodiment, at least one independently actuatable reservoir is customized for a specific biological subject. In an embodiment, at least one of the independent actuatable reservoirs is customized for a disease or condition of a specific biological subject. In an embodiment, the one or more independently actuatable reservoirs include one or more of an immune cell regulatory molecule, an antigen, a biological cell, a detectable indicator, or a surface antimicrobial agent.

In an embodiment, the antigen includes one or more of oxidized LDL cholesterol, HSP60, ApoB100, tumor-specific antigen, microbial capsular antigen, hepatitis B core antigen, hepatitis B e antigen, hepatitis B surface antigen, prostate-specific antigen, alphfetoprotein, carcinoembryonic antigen, CA-125, epithelial tumor antigen, tyrosinase, melanoma-associated antigen, abnormal product of Ras, abnormal product of p53, CALLA, MART-1/melana, gp100, GD-2, O-acetylated GD-3, GM-2, MUC-1, SOS1, AKAP protein, protein kinase C-binding protein, VRK1, KIAA1735, T7-1, T11-3, T11-9, CYFRA21-1, SCCA-1, SCCA-2, Orf73, NY-CO-45, NY-LU-12 variant A, ART1, NOVA2, CO-029, NY-BR-15, NY-BR-16, DUPAN-2, CA 19-9, CA 72-4, CA 195, CEA, GP120, SIV229, SIVE660, SHIV89.6P, E92, HCl, OKM5, FVIIIRAg, HLA-DR (Ia) antigens, OKM1, LFA-3, ESAT-6, CFP-10, Rv3871, CRA, RAP-2, MSP-2, AMA-1, GAD 65, HSP60, insulin peptide B9-23, or other antigen.

In an embodiment, an implantable system comprises a device (e.g., a microfluidics device) having a body structure including a surface with one or more sensors; and a plurality of independently manipulatable immune cell stimulus delivering substrates configured to deliver a patterned immune cell stimulus to the one or more regions proximate the surface of the body structure, the plurality of independently manipulatable immune cell stimulus delivering substrates defining at least a portion of the surface of the body structure.

In an embodiment, an implantable system comprises a device (such as a microfluidics device) having a body structure including a surface with one or more sensors; and a plurality of independently manipulatable immune cell stimulus delivering substrates configured to deliver a patterned immune cell stimulus to the one or more regions proximate the surface of the body structure, the plurality of independently manipulatable immune cell stimulus delivering substrates defining at least a portion of the surface of the body structure.

In an embodiment, microfluidics device comprises a body structure including a surface with one or more sensors; and a plurality of independently manipulatable immune cell stimulus delivering substrates configured to deliver a patterned immune cell stimulus to the one or more regions proximate the surface of the body structure, the plurality of independently manipulatable immune cell stimulus delivering substrates defining at least a portion of the surface of the body structure.

In an embodiment, a method comprises concurrently or sequentially delivering to one or more regions proximate the surface of an implantable microfluidics device a spatially patterned immune cell stimulus via a plurality of independently manipulatable immune cell stimulus delivering substrates configured to independently activate in response to a real-time detected parameter associated with a biological sample within the one or more regions proximate the surface of the microfluidics device.

In an embodiment, a method comprises concurrently or sequentially delivering to one or more regions proximate the surface of the microfluidics device a temporally patterned immune cell stimulus via a plurality of independently manipulatable immune cell stimulus delivering substrates configured to independently activate in response to a real-time detected parameter associated with at least one of biochemical information or biological cell information associated with a biological sample within one or more regions proximate the surface of the microfluidics device. In an embodiment, an implantable system (such as a fluid management system) comprises a device (e.g., a microfluidics device) having a body structure including a surface with one or more sensors; and a plurality of independently manipulatable immune cell stimulus delivering substrates configured to deliver a patterned immune cell stimulus to the one or more regions proximate the surface of the body structure, the plurality of independently manipulatable immune cell stimulus delivering substrates defining at least a portion of the surface of the body structure.

In an embodiment, an implantable device comprises a body structure having a surface including optionally one or more sensors; and one or more sealed reservoirs (optionally reversibly sealed configured to unseal in response to a reversibly sealed reservoir stimulus), and configured to direct an emitted biochemical or biological cell to one or more regions proximate the surface of the body structure and to deliver a patterned immune cell stimulus to the one or more regions proximate the surface of the body structure.

In an embodiment, a method of regulating an immune cell response from an at least partially implanted microfluidic device comprises selectively and actively releasing one or more biochemicals or one or more biological cells to one or more regions proximate the surface of an implanted portion of the device (e.g., microfluidic device) via one or more sealed reservoirs (optionally reversibly sealed), and delivering a patterned immune cell stimulus composition to the one or more regions proximate the surface of the implanted portion of the microfluidic device in response to an automatically detected parameter associated with a biological sample proximate the surface of the implanted portion of the microfluidic device via one or more sensors of the device.

In an embodiment, an implantable system comprises a device (e.g., a microfluidics device) having a body structure including a surface with one or more sensors; and a plurality of independently manipulatable immune cell stimulus delivering sealed reservoirs (optionally reversibly sealed) configured to deliver a patterned immune cell stimulus to the one or more regions proximate the surface of the body structure, the plurality of independently manipulatable immune cell stimulus delivering sealed reservoirs defining at least a portion of the surface of the body structure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 illustrates a partial view of an embodiment of a system described herein.

FIG. 2 illustrates a partial view of an embodiment of a system described herein.

FIG. 3 illustrates a partial view of an embodiment of a device described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Microfluidic devices (e.g., micro-electro-mechanical (MEMS) devices, lab-on-a-chip, lab-on-a-foil, thin film devices, flexible film devices, or the like), are useful for, among other things, miniaturization, integration, and automation of laboratory routines, including managing movement of fluids; directly detecting (e.g., assessing, calculating, evaluating, determining, gauging, identifying, measuring, monitoring, quantifying, resolving, sensing, or the like) mechanical, physical, or biochemical information (e.g., the presence of a biomarker, presence or absence of an immune cell stimulus, presence or absence of a biological cell (e.g., an immune cell), a disease state, or the like) associated with a biological subject; draining or collecting body fluids; as well as for administering therapeutics directly or via one or more immune cell; and activating or repressing an immune response (e.g., stimulating one or more antigen presenting cell, or one or more lymphocyte, or one or more NK cell).

Controlled immune responses, particularly of host immune cells, allows for tighter regulation of cells and related biochemical involved in an immune response. In particular, spatial and temporal regulation of activation or repression of certain immune cells enables directional control of the immune response to various diseases, whether as a prophylactic (e.g., vaccine) or therapeutic (e.g., response to one or more symptoms or tests positively indicating disease presence in a biological subject) treatment. For example, in an embodiment, a device or system disclosed herein functions as an artificial germinal center, or even an artificial lymph node (e.g., assembling multiple devices or multiple stacks or arrays of reservoirs or substrates that contain immune cell stimuli). For example, in an embodiment, a system or device disclosed herein is implanted as a supplement or replacement for lymph node function in subjects who have disease, who have had one or more lymph nodes removed, or who have low-functioning lymph nodes. In an example, a system or device disclosed herein is implanted as a supplement to an otherwise normally functioning lymph node, and is utilized for vaccination or other prophylactic address of disease. In an embodiment, the device is configured to be implanted in a biological subject proximate at least one of a burn, wound, tumor, surgery site, disease site, skin, or immunologically active region. In an embodiment, the immunologically active region includes at least one of a thymus, lymph node, tonsil, Peyer's patch, spleen, appendix, tonsil, adenoid, or bone marrow.

Accordingly, an aspect includes systems, devices, and methods, including a microfluidic device configured to, for example, detect (e.g., assess, calculate, evaluate, determine, gauge, identify, measure, monitor, quantify, resolve, sense, or the like) a biochemical or biological cell proximate the microfluidic device. A non-limiting example includes systems, devices, and methods including a microfluidic device configured to, for example, detect a biochemical or biological cell present in, for example, a biological specimen (e.g., tissue, biological fluid, target sample, disease site, or the like) proximate (e.g., on, near, or the like) a surface of the microfluidic device.

An aspect includes systems, devices, methods, and compositions for actively activating or repressing an immune response. In an embodiment, the immune response is related to, for example, detecting, treating, or preventing a disease or condition. An aspect includes systems, devices, and methods for managing movement of fluids; directly detecting and monitoring functions or conditions (e.g., mechanical, physical, physiological, or biochemical functions or conditions) associated with a biological subject; collecting body fluids; activating or repressing an immune response (e.g., from host cells of the biological subject) as well as for administering therapeutics as a prophylactic or responsive treatment. A non-limiting example includes systems, devices, and methods for actively recruiting, engaging, and/or repressing or activating one or more endogenous antigen presenting cells in the biological subject. Another non-limiting example includes systems, devices and methods for actively engaging and/or repressing or activating one or more antigen presenting cells provided by at least one reservoir or substrate of the device.

In an embodiment, a polymer matrix is included on the surface of the device for capture and/or release of biological cells or biochemicals from the biological subject. For example, attraction or repulsion of cells is regulated by the device by way of the type of cytokines present in the polymer matrix, or the concentration of cytokines in the matrix (e.g., concentration gradient is established by active or passive release of a concentration gradient that attracts or repels particular immune cells, such as antigen presenting cells, lymphocytes, or NK cells).

In an embodiment, a system of device described herein is implanted into a biological subject. In an embodiment, the device or system is either pre-loaded with a panel of immune cell stimuli (including one or more biological cells) or is loaded subsequent to implantation. In an embodiment, the device or system includes a general panel of immune cell stimuli (e.g., in response to disease or as a prophylactic such as a vaccine). In an embodiment, the device or system includes a personalized or customized panel of immune cell stimuli specifically selected for the particular health state or disease state of the biological subject.

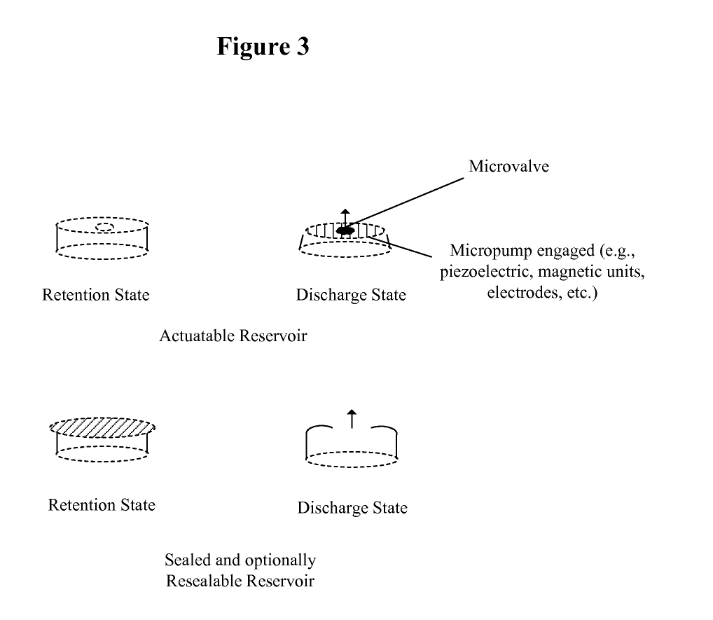

In an embodiment, at least one reservoir of the device (e.g., an actuatable or reversibly sealable reservoir) also includes one or more pumps and/or valves (e.g., micropumps or microvalves as shown in FIG. 3) for active control of delivery of small volumes contained in the reservoir. In an embodiment, the device further includes at least one computing device operably coupled to the micropump or microvalve and configured to actuate the micropump or microvalve between a reservoir discharge state and a reservoir retention state based on a comparison of a detected biological cell or biochemical to stored reference data.

In an embodiment, the active delivery of contents of the reservoir may operate by one or more of manual actuation, electrolysis, piezoelectric actuation, resistive heating, magnetic actuation, or by incorporation of reversible polymeric valves. In an embodiment, the pump is fabricated from one or more layers of elastic magnetic polymer membrane (e.g., polymethylsiloxane or similar polymer). Upon application of magnetic field (e.g., approximately 255 mT), the polymer membrane deforms, causing expulsion of the contents of the reservoir. See, for example, Stevenson, et al., Adv. Drug Delivery Rev., pp. 1590-1602 (2012), which is incorporated herein by reference. In an embodiment, the active delivery system includes a reservoir and/or valve made of a flexible membrane material such as parylene with two electrodes located on silicon in contact with the contents of the reservoir. Upon application of an electric field, gas is generated by electrolysis of the water present in the reservoir, which increases the pressure on the flexible membrane of the reservoir, forcing out the contents of the reservoir. In an embodiment, a piezo-actuated silicon micropump is utilized for active release of the contents of the reservoir. For example, a pump is fabricated from one or more layers of silicon and glass bonded with a piezoelectric ceramic disc and titanium fluid connectors, optionally with a reciprocating pumping membrane to guide flow of the contents of the reservoir away from the device. In an embodiment, a pump of the device includes at least one of piezoelectric disc actuator, electrostatic force, thermopneumatic force, shape memory alloy, electromagnetic, electrowetting, or acoustically excited oscillating bubble. See for example, Ryu, et al. JALA, pp. 163-171 (2010).

In an embodiment, a system or device includes an array of reservoirs (e.g., resealable or independently actuatable reservoirs, etc.) that can be actuated by electrochemical, electrothermal, or laser means, for example. For example, the contents of the reservoir are sealed in a reservoir in order to keep it isolated or in order for multiple different reservoirs to include different contents for administration in a specific sequence, or timing, etc. As described herein, release of contents of a reservoir of the device or system can be controlled for example, by a feedback loop (e.g., through sensors), by remote control, by direct activation due to a particular event, or by way of computer-implemented programming. Then, for example, initiation of release of the contents of the reservoir(s) is actively controlled by the application of the electrical or laser stimulus to create an opening in the sealing material, thereby releasing the contents of the reservoir. For example, the rate of release is passively controlled by the dissolution and diffusion of the contents of the reservoir, or actively controlled by continuous application of the electrical or other stimulus. For example, timing of delivery of multiple reservoirs of biochemicals or biological cells can be controlled by way of arrays of gold membrane capped reservoirs in silicon can be utilized such than when an electrical potential is applied to the gold cap in physiological levels of saline, the gold membrane is converted to a soluble gold salt and the contents of the reservoir are released. See Id.

Similarly, in an embodiment, a reservoir array can be operated electrothermally by utilizing metal membranes of gold, platinum, or titanium laminate for sealing reservoirs, for example, that are removed by resistive heating from an applied current. See Id.

In an embodiment, a system or device includes one or more immune cell stimuli that serve to activate or prime immune cells with a specific antigen, thereby enhancing immune defense. In an embodiment, the system or device attracts endogenous immune cells, such as macrophages, monocytes, NK cells, T cells, B cells, antigen presenting cells (e.g., dendritic cells), or epithelial cells, or precursors of any of these cells, with a chemical or other attractant (e.g., electrical, magnetic, thermal, etc.). In an embodiment, the system or device includes immune cells that are released for therapy of prophylactic treatment of disease. By exposing the immune cells to immune cell stimuli (such as specific antigens, target molecules, ligands, etc.), the immune response to a specific antigen can be elicited (e.g., viral or bacterial proteins), the immune response to a particular antigen can be dampened (e.g., self-antigens), or the immune response to a particular antigen can be regulated (e.g, first by increasing the response, then subsequently by decreasing the response).

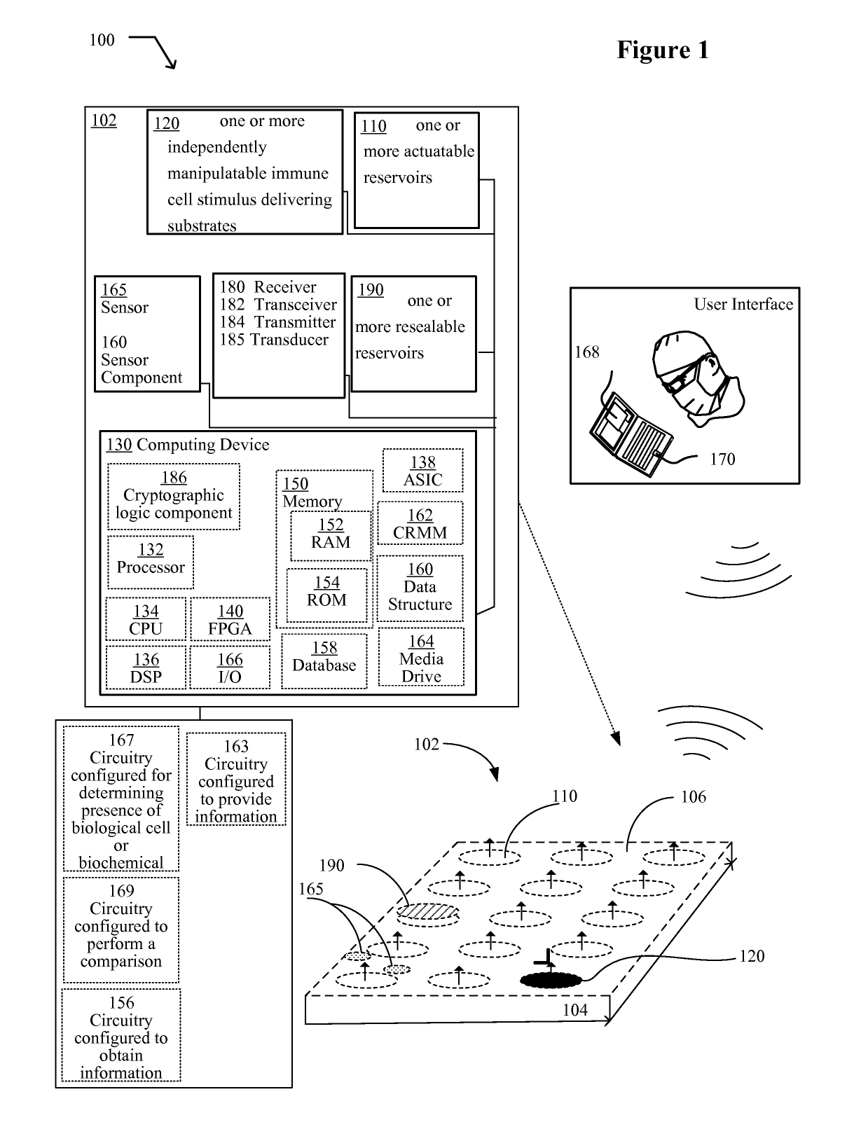

FIG. 1 shows a system 100 (e.g., a microfluidic system, an implantable microfluidic system, a partially implantable system, a system, or the like) in which one or more methodologies or technologies can be implemented such as, for example, actively inducing an immune response (e.g., activating or repressing an immune response), detecting, treating, or preventing a disease or condition, vaccinating a biological subject, or the like.

In an embodiment, the system 100 is configured to, among other things, provide an immune cell stimulus to a host cell of a biological subject (e.g., to one or more antigen presenting cells or one or more lymphocytes or one or more NK cells), or provide one or more biochemical (immune cell molecules such as cytokines, growth factors, or the like), or provide one or more biological cells (e.g., immune cells) managed by the system 100, or a biological sample proximate one or more components of the system 100. In an embodiment, the system 100 is configured to provide detectable indicators of one or more biochemical or biological cell, or provide one or more antimicrobial agents to the surface of the device or to the biological sample proximate the device. In this way, the antimicrobial agent can be utilized to either keep the surface of the microfluidic device clean of potential microbial infection, or release antimicrobial agents into the biological subject for preventative or responsive treatment of infection (e.g., local infection, or systemic infection transmitted by blood supply).

In an embodiment, the detectable indicator includes gold particles, magnetic particles, or a colorimetric dye.

In an embodiment, the patterned immune cell stimulus is configured to activate one or more of a dendritic cell, macrophage, B cell, follicular dendritic cell, Langerhans cell, or epithelial cell. In an embodiment, the patterned immune cell stimulus is configured to activate a biological cell expressing Major Histocompatibility Complex class II. In an embodiment, the patterned immune cell stimulus is configured to activate a biological cell from a subject including one or more of a mammal, bird, fish, reptile, or amphibian. In an embodiment, the patterned immune cell stimulus is configured to activate a biological cell from a human.

In an embodiment, the system 100 includes, among other things, at least one microfluidic device 102. In an embodiment, the microfluidic device 102 includes, among other things, a body structure 104 having a surface 106. In an embodiment, the system 100 is configured to provide an immune cell stimulus in the immediate vicinity of the device 102. For example, in an embodiment, the system 100 is configured to controllably deliver one or more immune cell stimuli to the surface 106 of the device 102 at a dose sufficient to modulate the activity of one or more biological cells (e.g., immune cells) in the immediate vicinity of a microfluidic device. In an embodiment, the microfluidic device 102 is configured to be implanted into a subject including one or more of a mammal, bird, fish, reptile, or amphibian. In an embodiment, the subject includes a human.

FIG. 1 shows various configurations of a system 100 in which one or more methodologies or technologies can be implemented. In an embodiment, the system 100 includes, among other things, at least one microfluidic device 102 including one or more independently manipulatable immune cell stimulus delivering substrates 120 and/or one or more actuatable reservoirs 110 and/or one or more resealable reservoirs 190. The one or more independently manipulatable immune cell stimulus delivering substrates 120 and/or one or more actuatable reservoirs 110 and/or one or more resealable reservoirs 190 can take a variety of shapes, configurations, and geometries including, but not limited to, cylindrical, conical, planar, parabolic, regular or irregular forms. In an embodiment, one or more independently manipulatable immune cell stimulus delivering substrates 120 and/or one or more actuatable reservoirs 110 and/or one or more resealable reservoirs 190 are formed from a single substrate or structure.

In an embodiment, a first immune cell stimulus (or stimuli) of the implantable device includes one or more biochemical (e.g., chemokine, cytokine, etc.) that is pre-loaded for mass manufacturing of the devices. In the same or another embodiment, a second immune cell stimulus (or stimuli) of the implantable device includes one or more biochemical or biological cell (e.g., an antigen) that has been personalized based on analysis of biological fluids or cells from the subject into which the device is to be implanted (e.g., blood sample, genetic sample, cell surface analysis, disease analysis). Thus, in this way a particular lot of manufactured devices includes a generalized immune cell stimulus array as well as at least one reservoir or substrate with a personalized immune cell stimulus present (contained therein, or attached to the surface, etc.).

In an embodiment, one or more independently manipulatable immune cell stimulus delivering substrates 120 and/or one or more actuatable reservoirs 110 and/or one or more resealable reservoirs 190 are configured to concurrently or sequentially provide one or more immune cell stimuli. In an embodiment, one or more independently manipulatable immune cell stimulus delivering substrates 120 and/or one or more actuatable reservoirs 110 and/or one or more resealable reservoirs 190 are configured to concurrently or sequentially provide at least a first immune cell stimulus and a second immune cell stimulus.

With regard to the one or more independently manipulatable immune cell stimulus delivering substrates 120, include an antigen emitting coating. In an embodiment, the one or more independently manipulatable immune cell stimulus delivering substrates 120, include an immune cell regulatory molecule emitting coating. In an embodiment, the one or more independently manipulatable immune cell stimulus delivering substrates 120 include a biological cell encapsulated or tethered to the surface. In an embodiment, the one or more independently manipulatable immune cell stimulus delivering substrates 120 include a detectable indicator emitting coating. In an embodiment, the one or more independently manipulatable immune cell stimulus delivering substrates 120 include a surface antimicrobial agent emitting coating. In an embodiment, the one or more independently manipulatable immune cell stimulus delivering substrates 120 include one or more coated nanoparticles. In an embodiment, the one or more independently manipulatable immune cell stimulus delivering substrates 120 include a surface material configured to emit the immune cell stimulus in the presence of a polymer-responsive stimulus. In an embodiment, the surface material includes at least one electroactive polymer, and the polymer-responsive stimulus includes an electric field. In an embodiment, the surface material includes at least one thermal-active polymer, and the polymer-responsive stimulus includes a heat field. In an embodiment, the surface material includes at least one magnetic-active polymer, and the polymer-responsive stimulus include a magnetic field. In an embodiment, the surface material includes at least one light-active polymer, and the polymer-responsive stimulus includes a light field.

In an embodiment, the surface of the body structure contains at least one biocompatible polymer or biocompatible metal. In an embodiment, the biocompatible metal includes at least one of titanium, copper, gold, or silver. In an embodiment, the biocompatible polymer includes at least one of polyethylene, polypropylene, polytetrafluroethylene, polymethylmethacrylate, ethylene-co-vinylacetate, polydimethylsiloxane, low molecular weight polydimethylsiloxane, polyethylene terephthalate, polysulphone, polyethyleneoxide, polyethyleneoxide-co-propyleneoxide, or polyvinylalcohol.

In an embodiment, the immune cell stimulus is enzymatically cleavable from the plurality of independently manipulatable immune cell stimulus delivering substrates. For example, the enzymatically cleavable immune cell stimulus includes an enzymatically cleavable antigen or enzymatically cleavable immune cell regulatory molecule. For example, an enzymatically cleavable immune cell stimulus includes an immune cell stimulus cleavable by at least one of a serine protease, an arginine protease, or the like.

In an embodiment, the immune cell stimulus includes an immune cell stimulus releasable by a pH-switch. In an embodiment, the pH-switch is configured to adsorb the immune cell stimulus to the surface at low pH and release it from the surface at high pH. In an embodiment, the low pH includes a pH of about 6 or less, about 5 or less, about 4 or less, about 3 or less, about 2 or less, or about 1 or less. In an embodiment, the high pH includes a pH of about 8 or higher, about 9 or higher, about 10 or higher. In an embodiment, the pH-switch is included as at least part of the surface of the body structure of the device. In an embodiment, the pH-switch includes one or more poly(methacrylic acid) chains combined with three-dimensional nanostructured silicon nanowire arrays.

With regard to the sealed (and optionally resealable) reservoirs 190, the sealed reservoirs (optionally reversibly sealed reservoirs) include at least one of a polymer, or a metal. In an embodiment, the polymer includes a conducting polymer. In an embodiment, the conducting polymer includes polyaniline or polypyrrole. In an embodiment, the polymer contains one or more of copper, nickel, gold, platinum, or silver. In an embodiment, the polymer contains a metal such as iron.

In an embodiment, the reversibly sealed reservoir stimulus includes at least one of electric field, pH, salinity, or magnetic field. In an embodiment, the one or more sealed reservoirs (optionally reversibly sealed reservoirs) are configured to deliver a spatially patterned immune cell stimulus. In an embodiment, the one or more sealed reservoirs are configured to deliver a temporally patterned immune cell stimulus. In an embodiment, the device further includes control circuitry operably coupled to the one or more sealed reservoirs and configured to control at least one of a spatial configuration parameter, or a temporal distribution parameter associated with the delivery of the patterned immune cell stimulus.

In an embodiment, the device further includes a computing device operably coupled to the one or more sealed reservoirs (optionally reversibly sealed reservoirs) and configured to control at least one of a spatial distribution, or a temporal distribution associated with the delivery of the patterned immune cell stimulus. In an embodiment, the one or more (optionally reversibly) sealed reservoirs include one or more of an immune cell regulatory molecule, an antigen, a biological cell, a detectable indicator, or a surface antimicrobial agent. In an embodiment, the one or more (optionally reversibly) sealed reservoirs include a plurality of spaced-apart (optionally reversibly) sealed reservoirs configured to deliver the immune cell stimulus in a temporally patterned distribution. In an embodiment, the device includes a plurality of spaced-apart (optionally reversibly) sealed reservoirs; and at least one computing device operably coupled to one or more of the plurality of spaced-apart (optionally reversibly) sealed reservoirs and configured to actuate one or more of the plurality of spaced-apart (optionally reversibly) sealed reservoirs between a reservoir discharge state and a reservoir retention state by inducing a reversibly sealed reservoir stimulus.

In an embodiment, the one or more sensors are configured to detect at least one biological cell or biochemical proximate the surface of the body structure; and at least one computing device operably coupled to one or more of the plurality of spaced-apart (optionally reversibly) sealed reservoirs and configured to actuate one or more of the plurality of spaced-apart (optionally reversibly) sealed reservoirs between a reservoir discharge state and a reservoir retention state based on a comparison of a detected biological cell or biochemical to stored reference data.

With continued reference to FIG. 1, in an embodiment the system 100 includes, among other things, at least one computing device 130 including one or more processors 132 (e.g., microprocessors), central processing units (CPUs) 134, digital signal processors (DSPs) 136, application-specific integrated circuits (ASICs) 138, field programmable gate arrays (FPGAs) 140, controllers, or the like, or any combinations thereof, and can include discrete digital or analog circuit elements or electronics, or combinations thereof. In an embodiment, the system 100 includes, among other things, one or more field programmable gate arrays 140 having a plurality of programmable logic components. In an embodiment, the system 100 includes, among other things, one or more application specific integrated circuits having a plurality of predefined logic components.

In an embodiment, at least one computing device 130 is operably coupled to one or more independently manipulatable immune cell stimulus delivering substrates 120 and/or one or more actuatable reservoirs 110 and/or one or more resealable reservoirs 190. In an embodiment, the system 100 includes one or more computing devices 130 configured to concurrently or sequentially operate multiple independently manipulatable immune cell stimulus delivering substrates 120 and/or one or more actuatable reservoirs 110 and/or one or more resealable reservoirs 190. In an embodiment the computing device 130 comprises at least one controller. In an embodiment, at least one computing device 130 is operably coupled to one or more actuatable reservoirs 110. In an embodiment, one or more of the actuatable reservoirs 110 are configured for selective actuation via one or more computing devices 130.

In an embodiment, the system 100 includes one or more microfluidic devices 102 including, among other things, one or more receivers 180, transceivers 182, or transmitters 184. In an embodiment, at least one of the one or more receiver 180, transceivers 182, and transmitters 184, can be, for example, wirelessly coupled to a computing device 130 that communicates with a control unit of the system 100 via wireless communication. In an embodiment, the transmitter 184 is configured to send information based at least in part on a detected biological cell or biochemical. In an embodiment, the transmitter 184 is configured to send a request for transmission of at least one of a command, an authorization, an update, or a code.

In an embodiment, at least one of the one or more receivers 180 and transceivers 182 is configured to acquire information associated with a set of targets, markers, or the like for detection. In an embodiment, at least one of the one or more receivers 180 and transceivers 182 is configured to acquire information associated with a set of physiological characteristic for detection. In an embodiment, at least one of the one or more receivers 180 and transceivers 182 is configured to acquire information associated with one or more physiological characteristics for detection. In an embodiment, at least one of the one or more receivers 180 and transceivers 182 is configured to acquire information associated with one or more physiological characteristics for detection.

In an embodiment, at least one receiver 180 is configured to acquire information based at least in part on whether a detected biological cell or biochemical proximate the surface of the body structure satisfies a target condition. In an embodiment, the target condition includes at least one of a type of biological cell, type of biochemical, level of a biochemical, timing of delivery of an immune cell stimulus, response based on the type of surface of the body structure of the device, temporal delivery of an immune cell stimulus, or spatial delivery of an immune cell stimulus. In an embodiment, the type of biological cell includes the type of immune cell. In an embodiment, the type of immune cell includes at least one of an antigen presenting cell, lymphocyte, or NK cell.

In an embodiment, the at least one receiver 180 is configured to acquire information associated with delivery of the contents of the one or more reservoirs (e.g., resealable, actuatable, etc.). In an embodiment, the at least one receiver 180 is configured to acquire data. In an embodiment, the at least one receiver 180 is configured to receive stored reference data. In an embodiment, the at least one receiver 180 is configured to acquire software. In an embodiment, the at least one receiver 180 is configured to receive data from one or more implantable sensors remote from the implantable device. In an embodiment, the at least one receiver 180 is configured to receive data from one or more input/output devices.

In an embodiment, at least one receiver 180 is configured to receive a wireless signal. In an embodiment, the receiver 180 is configured to receive a signal from a remote source (e.g., computing device, etc.). In an embodiment, the receiver 180 is configured to receive a signal from a pre-programmed sequence of activation and/or actuation of the various reservoirs or substrates of the device.

In an embodiment, at least one receiver 180 is configured to acquire information associated with a delivery of an immune cell stimulus. In an embodiment, the at least one receiver 180 is configured to acquire data. In an embodiment, the at least one receiver 180 is configured to acquire software. In an embodiment, the at least one receiver 180 is configured to receive data from one or more distal sensors. In an embodiment, the at least one receiver 180 is configured to receive stored reference data. In an embodiment, the at least one receiver 180 is configured to acquire at least one of instructions, instructions associated with a delivery of an immune cell stimulus, instructions associated with a delivery of an immune cell stimuli, information associated with a biological sample, instructions associated with a biological fluid, instructions associated with a disease state, or the like.

In an embodiment, the system 100 includes one or more receivers 180 configured to acquire spectral information (e.g., radio frequency (RF) information) emitted by an in vivo biological sample. In an embodiment, the one or more receivers 180 include one or more of analog-to-digital converters, signal amplifier, matching networks, oscillators, power amplifiers, RF receive coils, RF synthesizers, or signal filters. In an embodiment, the system 100 includes one or more transceivers 182 (e.g., RF transceivers) configured to generate RF excitation pulses that interacts with, for example, an in vivo target.

In an embodiment, the system 100 includes control circuitry operably coupled to the one or more independently manipulatable immune cell stimulus delivering substrates 120 and/or one or more actuatable reservoirs 110 and/or one or more resealable reservoirs 190 and configured to control at least one of a spaced-apart configuration parameter, an immune cell stimulus spatial distribution parameter, or an immune cell temporal distribution parameter associated with the delivery of the patterned immune cell stimulus. In an embodiment, at least one computing device 130 is operably coupled to one or more independently manipulatable immune cell stimulus delivering substrates 120 and/or one or more actuatable reservoirs 110 and/or one or more resealable reservoirs 190 and configured to control at least one of a delivery regiment, a spatial distribution, or a temporal distribution associated with the delivery of the patterned immune cell stimulus. In an embodiment, the one or more computing devices 130 are configured to select and activate at least one of the plurality of one or more independently manipulatable immune cell stimulus delivering substrates 120 and/or one or more actuatable reservoirs 110 and/or one or more resealable reservoirs 190 in response to a scheduled program, an external command, a history of a previous immune cell stimulus (in general, or in particular), or a history of a previous actuation.

In an embodiment, the system 100 includes one or more microfluidic devices 102 including for example, but not limited to, circuitry for providing information. In an embodiment, the circuitry for providing information includes circuitry for providing status information regarding the implantable device. In an embodiment, the circuitry for providing information includes circuitry for providing information regarding at least one characteristic associated with a biological subject. For example, in an embodiment, the circuitry for providing information includes circuitry for providing information regarding at least one characteristic associated with a tissue or biological fluid proximate the microfluidic device 102. In an embodiment, the circuitry for providing information includes circuitry for providing information regarding at least one physiological characteristic associated with the biological subject. In an embodiment, the circuitry for providing information includes circuitry for providing information regarding at least one characteristic associated with a biological sample of the biological subject. In an embodiment, the circuitry for providing information includes circuitry for providing information regarding at least one characteristic associated with a tissue proximate the one or more fluid-flow passageways 110. In an embodiment, the system 100 includes one or more microfluidic devices 102 including for example, but not limited to, circuitry for transmitting information. In an embodiment, the at least one transmitter 184 is configured to send information based at least in part on a detected characteristic associated with an immune cell (e.g., an antigen presenting cell, lymphocyte, or other immune cell) received from the surface of the device 110. In an embodiment, the at least one transmitter 184 is configured to send a request for transmission of at least one of data, a command, an authorization, an update, or a code. In an embodiment, the at least one data, command, authorization, update, or code relates to modulation of an immune cell (e.g., antigen presenting cell, lymphocyte, etc.). In an embodiment, the modulation of the immune cell results in the activation or repression of an immune response. Thus, in an embodiment the modulation relates to an increase in an immune response. In another embodiment, the modulation relates to a decrease in an immune response.

In an embodiment, the system 100 includes one or more microfluidic devices 102 including for example, but not limited to, one or more cryptographic logic components 186. In an embodiment, at least one of the one or more cryptographic logic components 186 is configured to implement at least one cryptographic process, or cryptographic logic, or combinations thereof. Non-limiting examples of a cryptographic process include one or more processes associated with cryptographic protocols, decryption protocols, encryption protocols, regulatory compliance protocols (e.g., FDA regulatory compliance protocols, or the like), regulatory use protocols, authentication protocols, authorization protocols, treatment regimen protocols, activation protocols, encryption protocols, decryption protocols, or the like. Non-limiting examples of a cryptographic logic include one or more crypto-algorithms signal-bearing media, crypto controllers (e.g., crypto-processors), cryptographic modules (e.g., hardware, firmware, or software, or combinations thereof for implementing cryptographic logic, or cryptographic processes), or the like.

In an embodiment, the cryptographic logic component 186 is configured to implement at least one cryptographic process or cryptographic logic. In an embodiment, the cryptographic logic component 186 is configured to implement one or more processes associated with at least one of a cryptographic protocol, a decryption protocol, an encryption protocol, a regulatory compliance protocol, a regulatory use protocol, an authentication protocol, an authorization protocol, a delivery protocol, an activation protocol, an encryption protocol, or a decryption protocol. In an embodiment, the cryptographic logic component 186 includes one or more crypto-algorithms, signal-bearing media, crypto controllers, or cryptographic modules.

In an embodiment, the cryptographic logic component 186 is configured to generate information associated with at least one of an authentication protocol, an authorization protocol, a delivery protocol (e.g., a sterilizing energy stimulus delivery protocol), an activation protocol, an encryption protocol, or a decryption protocol. In an embodiment, the cryptographic logic component 186 is configured to generate information associated with at least one of an authorization instruction, an authentication instruction, a prescription dosing instruction, a sterilizing energy stimulus administration instruction, or a prescribed regimen instruction.

In an embodiment, the cryptographic logic component 186 is configured to generate information associated with at least one of an instruction stream, an encrypted data stream, an authentication data stream, or an authorization data stream. In an embodiment, the cryptographic logic component 186 is configured to generate information associated with at least one of an activation code, an error code, a command code, or an authorization code. In an embodiment, the cryptographic logic component 186 is configured to generate information associated with at least one of a cryptographic protocol, a decryption protocol, an encryption protocol, a regulatory compliance protocol, or regulatory use protocol.

In an embodiment, the microfluidic device 102 is, for example, wirelessly coupled to a computing device 130 that communicates with the microfluidic device 102 via wireless communication. Non-limiting examples of wireless communication include optical connections, ultraviolet connections, infrared, BLUETOOTH®, Internet connections, radio, network connections, or the like.

In an embodiment, the microfluidic device 102 includes at least one computing device 130 configured to control one or more parameter associated with the operation of the microfluidic device 102. For example, in an embodiment, the microfluidic device 102 includes at least one computing device 130 operably coupled to one or more independently manipulatable immune cell stimulus delivering substrates 120 and/or one or more actuatable reservoirs 110 and/or one or more resealable reservoirs 190 and configured to control at least one parameter associated with the delivery of the immune cell stimulus. In an embodiment, the at least one computing device 130 is configured to control at least one of a duration time, amount, type, delivery location, programmed delivery (e.g., space-time sensitive) temporal-pattern stimulation configuration associated with delivery of the immune cell stimulus, or spatial-pattern stimulation configuration associated with the delivery of the immune cell stimulus.

In an embodiment, the system 100 includes, among other things, one or more electronic memories 150 that, for example, store instructions or data, for example, volatile memory (e.g., Random Access Memory (RAM) 152, Dynamic Random Access Memory (DRAM), or the like), non-volatile memory (e.g., Read-Only Memory (ROM) 154, Electrically Erasable Programmable Read-Only Memory (EEPROM), Compact Disc Read-Only Memory (CD-ROM), or the like), persistent memory, or the like. Further non-limiting examples of one or more memories 150 include Erasable Programmable Read-Only Memory (EPROM), flash memory, or the like. Various components of the microfluidic device 102 (e.g., memories 150, processors 132, or the like) can be operably coupled to each other via one or more instruction, data, or power buses 156.

In an embodiment, the system 100 includes, among other things, one or more databases 158. In an embodiment, a database 158 includes spectral information configured as a physical data structure. In an embodiment, a database 158 includes at least one of inflammation indication parameter data, infection indication parameter data, diseased tissue indication parameter data, immune cell stimuli to be delivered or previously delivered (e.g., prior antigen presenting cell stimulation), vaccine delivery, or the like. In an embodiment, a database 158 includes at least one of personalized health history, public health record, or the like. In an embodiment, a database 158 includes at least one of stored reference data such as infection marker data, inflammation marker data, vaccination marker data, a systemic inflammatory response syndrome data, sepsis marker data, chronic immune response data, or the like.

In an embodiment, a database 158 includes information associated with a disease state of a biological subject. In an embodiment, a database 158 includes information associated with the biological subject's personalized health record. In an embodiment, a database 158 includes information associated with a public medical or public health record.

In an embodiment, the system 100 is configured to compare an input associated with at least one characteristic associated with a biological subject to a database 158 of stored reference values, and to generate a response based in part on the comparison. In an embodiment, the system 100 is configured to compare an input associated with at least one physiological characteristic associated with a biological subject to a database 158 of stored reference values, and to generate a response based in part on the comparison.

In an embodiment, the at least one characteristic associated with a biological subject includes real-time detected information associated with a sample (e.g., tissue, biological fluid, infections agent, biomarker, or the like) proximate the microfluidic device 102. In an embodiment, the at least one characteristic associated with a biological subject includes real-time detected information associated with a sample (e.g., a biological fluid) received proximate the surface of the device.

In an embodiment, the system 100 is configured to compare an input associated with at least one characteristic associated with a biological sample proximate the microfluidic device 102 (e.g., received on or near the surface of the body structure 104, or the like) to a database 158 of stored reference values, and to generate a response based in part on the comparison. In an embodiment, the response includes at least one of a visual representation, an audio representation (e.g., an alarm, an audio waveform representation of a tissue region, or the like), a haptic representation, and a tactile representation (e.g., a tactile diagram, a tactile display, a tactile graph, a tactile interactive depiction, a tactile model (e.g., a multidimensional model of an infected tissue region, or the like), a tactile pattern (e.g., a refreshable Braille display), a tactile-audio display, a tactile-audio graph, or the like). In an embodiment, the response includes generating at least one of a visual, an audio, a haptic, or a tactile representation of biological sample information (e.g., biological fluid information, biological cell information, or biochemical information related to a particular tissue or cell, or the like). In an embodiment, the response includes generating at least one of a visual, an audio, a haptic, or a tactile representation of at least one physical or biochemical characteristic associated with a biological subject.

In an embodiment, the response includes initiating one or more treatment protocols. In an embodiment, the response includes delivering an immune cell stimulus. In an embodiment, the response includes concurrently or sequentially delivering an energy stimulus and an immune cell stimulus.

In an embodiment, the response includes at least one of a response signal, a control signal, a change to an immune cell stimulus parameter (e.g., an antigen presenting cell stimulus, lymphocyte stimulus), or the like.

In an embodiment, the response includes at least one of a change to an immune cell stimulus spatial pattern parameter, a change to an immune cell stimulus temporal parameter, or the like.

In an embodiment, the response includes at least one of activating an authorization protocol, activating an authentication protocol, activating a software update protocol, activating a data transfer protocol, or activating a diagnostic protocol. In an embodiment, the response includes sending information associated with at least one of an authentication protocol, an authorization protocol, a delivery protocol, an activation protocol, an encryption protocol, or a decryption protocol.

In an embodiment, a database 158 includes at least one of stored reference data such as characteristic biological sample (e.g., type, number, expression profile, etc. of an antigen presenting cell or lymphocyte/NK cell) component data, characteristic blood component data (e.g., cell type, cell expression of proteins whether surface or secreted proteins, biochemical type, or the like), characteristic tissue data, or the like.

Referring to FIG. 1, in an embodiment, the system 100 includes, among other things, one or more computer-readable media drives 164, interface sockets, Universal Serial Bus (USB) ports, memory card slots, or the like, or one or more input/output components 166 such as, for example, a graphical user interface 168, a display, a keyboard 170, a keypad, a trackball, a joystick, a touch-screen, a mouse, a switch, a dial, or the like, and any other peripheral device. In an embodiment, the system 100 includes one or more user input/output components 166 that operably couple to at least one computing device 130 to control (electrical, electromechanical, software-implemented, firmware-implemented, or other control, or combinations thereof) at least one parameter associated with the immune cell stimulus delivery associated with one or more independently manipulatable immune cell stimulus delivering substrates 120 and/or one or more actuatable reservoirs 110 and/or one or more resealable reservoirs 190.

In an embodiment, the system 100 includes, among other things, one or more modules optionally operable for communication with one or more input/output components 166 that are configured to relay user output and/or input. In an embodiment, a module includes one or more instances of electrical, electromechanical, software-implemented, firmware-implemented, or other control devices. Such devices include one or more instances of memory 150, computing devices 130, ports, valves, fuses, antifuses, antennas, power, or other supplies; logic modules or other signaling modules; gauges or other such active or passive detection components; or piezoelectric transducers, shape memory elements, micro-electro-mechanical system (MEMS) elements, or other actuators.

The computer-readable media drive 164 or memory slot can be configured to accept signal-bearing medium (e.g., computer-readable memory media, computer-readable recording media, or the like). In an embodiment, a program for causing the system 100 to execute any of the disclosed methods can be stored on, for example, a computer-readable recording medium (CRMM) 162, a signal-bearing medium, or the like. Non-limiting examples of signal-bearing media include a recordable type medium such as a magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transmission logic, reception logic, etc.), etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

In an embodiment, the system 100 includes signal-bearing media in the form of one or more logic devices (e.g., programmable logic devices, complex programmable logic device, field-programmable gate arrays, application specific integrated circuits, or the like) comprising, for example, a data structure 160 including one or more look-up tables. In an embodiment, the system 100 includes, among other things, non-transitory signal-bearing media having sample information (e.g., biological sample information, reference information for location of a particular immune cell stimulus on the microfluidic device) configured as a data structure 160. In an embodiment, the data structure 160 includes at least one of immune response information, antigen level information, biological cell density (e.g., for general or specific immune cells such as antigen presenting cells, or lymphocytes, macrophages, etc.), infection indication information, inflammation indication information, diseased state indication information, or diseased tissue indication information.

Referring further to FIG. 1, in an embodiment, the system 100 includes, among other things, at least one sensor component 160. In an embodiment, the microfluidic device 102 includes at least one sensor component 160. In an embodiment, the sensor component 160 is configured to detect (e.g., assess, calculate, evaluate, determine, gauge, measure, monitor, quantify, resolve, sense, or the like) at least one characteristic (e.g., a spectral characteristic, a spectral signature, a physical quantity, a relative quantity, an environmental attribute, a physiologic characteristic, or the like) associated with a biological subject. In an embodiment, the sensor component 160 is configured to perform a real-time comparison of a parameter associated with a biological sample proximate the microfluidic device 102 to stored reference data and to generate a response based on the comparison. For example, in an embodiment the sensor component is configured to sense the presence or absence of a biological cell or particular biochemical, or the changing status of presence/absence of the target cell or biochemical.

In an embodiment, the sensor component 160 is operably coupled to one or more computing device 130. In an embodiment, at least one computing device 130 is operably coupled to the sensor component 160 and configured to process an output associated with one or more sensed parameters. In an embodiment, at least one computing devices 130 is configured to concurrently or sequentially operate multiple sensor components 160. In an embodiment, the sensor component 160 includes a computing device 130 configured to process sensed parameter information and configured to cause the storing of the parameter information in a data storage medium. In an embodiment, the sensor component 160 includes a component identification code and is configured to implement instructions addressed to the sensor component 160 according to the component identification code.

In an embodiment, the sensor component 160 includes one or more surface plasmon resonance sensors. For example, in an embodiment, the sensor component 160 includes one or more localized surface plasmon resonance sensors. In an embodiment, the sensor component 160 includes a light transmissive support and a reflective metal layer. In an embodiment, the sensor component 160 includes a wavelength-tunable surface plasmon resonance sensor. In an embodiment, the sensor component 160 includes a surface plasmon resonance microarray sensor having a wavelength-tunable metal-coated grating. In an embodiment, the sensor component 160 includes a surface plasmon resonance microarray sensor having an array of micro-regions configured to capture target molecules (e.g., biochemicals or biological cells, including biological cell receptors). See, for example, Ouellet, et al. Lab Chip, 2010, 10(5): 581-8 (Abstract), which is incorporated herein by reference. In an embodiment, parallel surface plasmon resonance imaging arrays are utilized as part of the device to quantify binding affinities or concentrations of target molecules (such as a biochemical disclosed herein, cell surface receptor or secreted cell protein, etc.). See, Id.

In an embodiment, the sensor component 160 includes one or more electrochemical transducers, optical transducers, piezoelectric transducers, or thermal transducers. For example, in an embodiment, the sensor component 160 includes one or more transducers configured to detect acoustic waves associated with changes in a biological mass present proximate a surface of the body structure 104.

In an embodiment, the sensor component 160 includes one or more thermal detectors, photovoltaic detectors, or photomultiplier detectors. In an embodiment, the sensor component 160 includes one or more charge-coupled devices, complementary metal-oxide-semiconductor devices, photodiode image sensor devices, whispering gallery mode micro cavity devices, or scintillation detector devices. In an embodiment, the sensor component 160 includes one or more ultrasonic transducers. In an embodiment, the sensor component 160 includes at least one of a charge-coupled device, a complementary metal-oxide-semiconductor device, a photodiode image sensor device, a Whispering Gallery Mode (WGM) micro cavity device, and a scintillation detector device.

In an embodiment, the sensor component 160 includes at least one of an imaging spectrometer, a photo-acoustic imaging spectrometer, a thermo-acoustic imaging spectrometer, or a photo-acoustic/thermo-acoustic tomographic imaging spectrometer. In an embodiment, the sensor component 160 includes at least one of a thermal detector, a photovoltaic detector, or a photomultiplier detector.

In an embodiment, the sensor component 160 includes one or more density sensors. In an embodiment, the sensor component 160 includes one or more optical density sensors. In an embodiment, the sensor component 160 includes one or more refractive index sensors. In an embodiment, the sensor component 160 includes one or more fiber optic refractive index sensors.

In an embodiment, the sensor component 160 includes one or more acoustic biosensors, amperometric biosensors, calorimetric biosensors, optical biosensors, or potentiometric biosensors. In an embodiment, the sensor component 160 includes one or more fluid flow sensors. In an embodiment, the sensor component 160 includes one or more differential electrodes, biomass sensors, immunosensors, or the like. In an embodiment, the sensor component 160 includes one or more one-, two-, or three-dimensional photodiode arrays.

In an embodiment, the sensor component 160 includes a biological molecule capture layer having an array of different binding molecules that specifically bind one or more target molecules. In an embodiment, the sensor component 160 includes one or more computing devices 130 operably coupled to one or more sensors.

In an embodiment, the sensor component 160 is configured to detect at least one characteristic associated with a biological subject. In an embodiment, the sensor component 160 is configured to detect at least one characteristic associated with a biological specimen proximate a surface of the microfluidic device 102. For example, in an embodiment, the sensor component 160 is configured to detect at least one characteristic associated with a tissue proximate the microfluidic device 102. For example, in an embodiment, the sensor component 160 is configured to detect at least one biological cell (e.g., an immune cell, more specifically an antigen presenting cell or lymphocyte) proximate the microfluidic device.

In an embodiment, the at least one characteristic includes at least one parameter associated with a medical state (e.g., medical condition, disease state, disease attributes, etc.). For example, oxygen radicals, cytotoxic factors, and growth factors can also be released to fight pathogen infection or to facilitate tissue healing. This cascade of biochemical events propagates and matures the inflammatory response, involving the local vascular system, the immune system, and various cells within the injured tissue. Under normal circumstances, through a complex process of mediator-regulated pro-inflammatory and anti-inflammatory signals, the inflammatory response eventually resolves itself and subsides. For example, the transient and localized swelling associated with a cut is an example of an acute inflammatory response.

However, in certain cases resolution does not occur as expected. Prolonged inflammation, known as chronic inflammation, leads to a progressive shift in the type of cells present at the site of inflammation and is characterized by simultaneous destruction and healing of the tissue from the inflammatory process, as directed by certain mediators. Rheumatoid arthritis is an example of a disease associated with persistent and chronic inflammation.

In an embodiment, the at least one characteristic includes at least one parameter associated with an infection marker (e.g., an infectious agent marker), an inflammation marker, an infective stress marker, a systemic inflammatory response syndrome marker, or a sepsis marker. Non-limiting examples of infection makers, inflammation markers, or the like may be found in, for example, Imam et al., Radiotracers for imaging of infection and inflammation—A Review, World J. Nucl. Med. 40-55 (2006), which is incorporated herein by reference. Non-limiting characteristics associated with an infection marker, an inflammation marker, an infective stress marker, a systemic inflammatory response syndrome marker, or a sepsis marker include at least one of an inflammation indication parameter, an infection indication parameter, a diseased state indication parameter, or a diseased tissue indication parameter.

In an embodiment, the at least one characteristic includes at least one of a tissue water content, an oxy-hemoglobin concentration, a deoxyhemoglobin concentration, an oxygenated hemoglobin absorption parameter, a deoxygenated hemoglobin absorption parameter, a tissue light scattering parameter, a tissue light absorption parameter, a hematological parameter, or a pH level.

In an embodiment, the at least one characteristic includes at least one hematological parameter. Non-limiting examples of hematological parameters include an albumin level, a blood urea level, a blood glucose level, a globulin level, a hemoglobin level, erythrocyte count, a leukocyte count, lymphocyte count, antigen presenting cell count, NK cell count, level of expression of a cell surface receptor or ligand, level of a secreted cell protein, number of cells expressing or secreting a particular protein, or the like. In an embodiment, the infection marker includes at least one parameter associated with a red blood cell count, a lymphocyte level, a leukocyte count, a myeloid cell count, an erythrocyte sedimentation rate, or a C-reactive protein level. In an embodiment, the at least one characteristic includes at least one parameter associated with a cytokine plasma level or an acute phase protein plasma level. In an embodiment, the at least one characteristic includes at least one parameter associated with a leukocyte level.

Non-limiting examples of detectable blood components include erythrocytes, leukocytes (e.g., basophils, granulocytes, eosinophils, monocytes, macrophages, lymphocytes, neutrophils, or the like), thrombocytes, acetoacetate, acetone, acetylcholine, adenosine triphosphate, adrenocorticotrophic hormone, alanine, albumin, aldosterone, aluminum, amyloid proteins (non-immunoglobulin), antibodies, apolipoproteins, ascorbic acid, aspartic acid, bicarbonate, bile acids, bilirubin, biotin, blood urea Nitrogen, bradykinin, bromide, cadmium, calciferol, calcitonin (ct), calcium, carbon dioxide, carboxyhemoglobin (as HbcO), cell-related plasma proteins, cholecystokinin (pancreozymin), cholesterol, citric acid, citrulline, complement components, coagulation factors, coagulation proteins, complement components, c-peptide, c-reactive protein, creatine, creatinine, cyanide, 11-deoxycortisol, deoxyribonucleic acid, dihydrotestosterone, diphosphoglycerate (phosphate), or the like.

Further non-limiting examples of detectable blood components include dopamine, enzymes, epidermal growth factor, epinephrine, ergothioneine, erythrocytes, erythropoietin, folic acid, fructose, furosemide glucuronide, galactoglycoprotein, galactose (children), gamma-globulin, gastric inhibitory peptide, gastrin, globulin, α-1-globulin, α-2-globulin, α-globulins, β-globulins, glucagon, glucosamine, glucose, immunoglobulins (antibodies), lipase p, lipids, lipoprotein (sr 12-20), lithium, low-molecular weight proteins, lysine, lysozyme (muramidase), α-2-macroglobulin, γ-mobility (non-immunoglobulin), pancreatic polypeptide, pantothenic acid, para-aminobenzoic acid, parathyroid hormone, pentose, phosphorated, phenol, phenylalanine, phosphatase, acid, prostatic, phospholipid, phosphorus, prealbumin, thyroxine-binding, proinsulin, prolactin (female), prolactin (male), proline, prostaglandins, prostate specific antigen, protein, protoporphyrin, pseudoglobulin I, pseudoglobulin II, purine, pyridoxine, pyrimidine nucleotide, pyruvic acid, CCL5 (RANTES), relaxin, retinol, retinol-binding protein, riboflavin, ribonucleic acid, secretin, serine, serotonin (5-hydroxytryptamine), silicon, sodium, solids, somatotropin (growth hormone), sphingomyelin, succinic acid, sugar, sulfates, inorganic, sulfur, taurine, testosterone (female), testosterone (male), triglycerides, triiodothyronine, tryptophan, tyrosine, urea, uric acid, water, miscellaneous trace components, or the like.

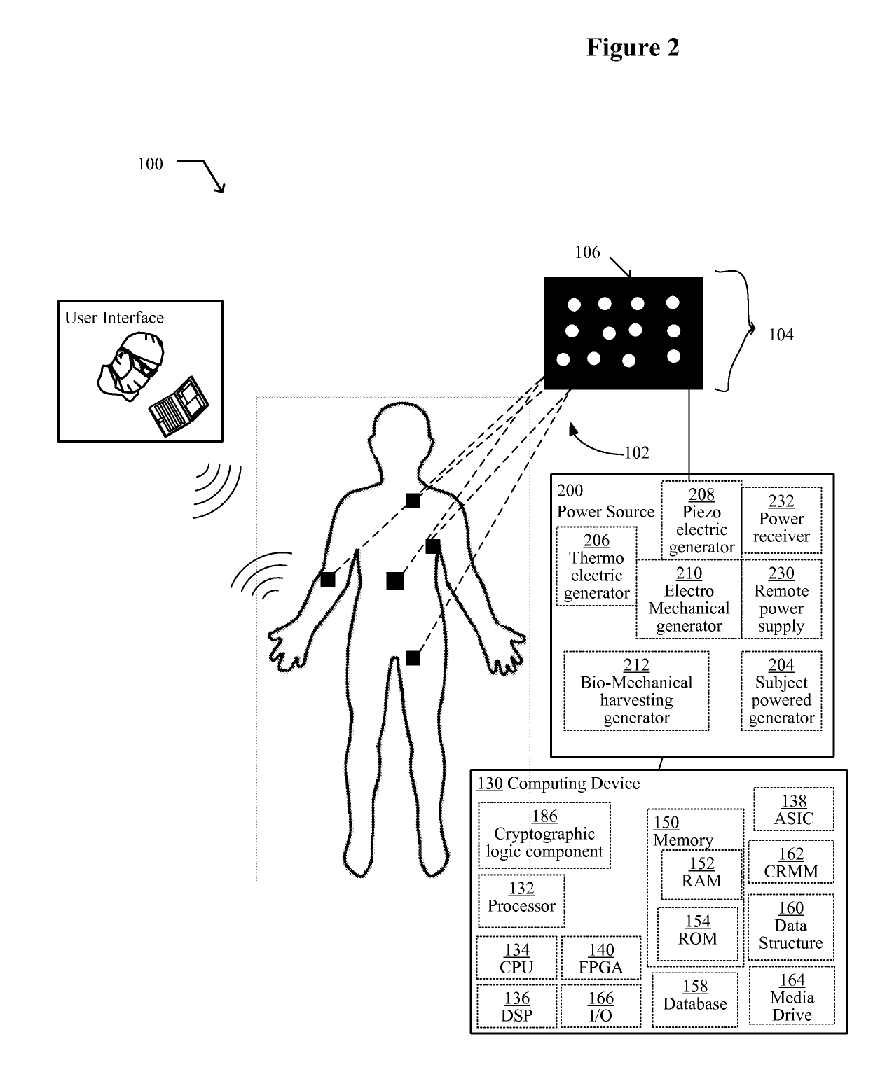

Referring to FIG. 2, in an embodiment the system 100 includes, among other things, one or more power sources 200. In an embodiment, the microfluidic device 102 includes one or more power sources 200. In an embodiment, the power source 200 is electromagnetically, magnetically, acoustically, optically, inductively, electrically, or capacitively coupled to at least one of the reservoirs or substrate locations, the computing device 130, and the sensor component 160. Non-limiting examples of power sources 200 examples include one or more button cells, chemical battery cells, a fuel cell, secondary cells, lithium ion cells, micro-electric patches, nickel metal hydride cells, silver-zinc cells, capacitors, super-capacitors, thin film secondary cells, ultra-capacitors, zinc-air cells, or the like. Further non-limiting examples of power sources 200 include one or more batteries, generators (e.g., electrical generators, thermo energy-to-electrical energy generators, mechanical-energy-to-electrical energy generators, micro-generators, nano-generators, or the like) such as, for example, thermoelectric generators, piezoelectric generators, electromechanical generators, biomechanical-energy harvesting generators, or the like. In an embodiment, the power source 200 includes at least one rechargeable power source. In an embodiment, the power source 200 is carried by the microfluidic device 102. In an embodiment, the microfluidic device 102 can include, among other things, at least one of a battery, a capacitor, or a mechanical energy store (e.g., a spring, a flywheel, or the like).

In an embodiment, the power source 200 is configured to wirelessly receive power from a remote power supply 230. In an embodiment, the microfluidic device 102 includes one or more power receivers 232 configured to receive power from an in vivo or ex vivo power source. In an embodiment, the power source 200 is configured to wirelessly receive power via at least one of an electrical conductor or an electromagnetic waveguide. In an embodiment, the power source 200 includes one or more power receivers 232 configured to receive power from an in vivo or ex vivo power source. In an embodiment, the in vivo power source includes at least one of a thermoelectric generator, a piezoelectric generator, a microelectromechanical systems generator, or a biomechanical-energy harvesting generator.

In an embodiment, the microfluidic device 102 includes one or more generators configured to harvest mechanical energy from for example, acoustic waves, mechanical vibration, blood flow, or the like. For example, in an embodiment, the power source 200 includes at least one of a biological-subject (e.g., human)-powered generator 204, a thermoelectric generator 206, piezoelectric generator 208, electromechanical generator 210 (e.g., a microelectromechanical systems (MEMS) generator, or the like), biomechanical-energy harvesting generator 212, or the like.

In an embodiment, the biological-subject-powered generator 204 is configured to harvest thermal energy generated by the biological subject. In an embodiment, the biological-subject-powered generator 204 is configured to harvest energy generated by the biological subject using at least one of a thermoelectric generator 206, piezoelectric generator 208, electromechanical generator 210 (e.g., a microelectromechanical systems (MEMS) generator, or the like), biomechanical-energy harvesting generator 212, or the like. For example, in an embodiment, the biological-subject-powered generator 204 includes one or more thermoelectric generators 206 configured to convert heat dissipated by the biological subject into electricity. In an embodiment, the biological-subject-powered generator 204 is configured to harvest energy generated by any physical motion or movement (e.g., walking,) by biological subject. For example, in an embodiment, the biological-subject-powered generator 204 is configured to harvest energy generated by the movement of a joint within the biological subject. In an embodiment, the biological-subject-powered generator 204 is configured to harvest energy generated by the movement of a fluid (e.g., biological fluid) within the biological subject.

In an embodiment, the one or more sensors are configured to detect at least one biological cell or biochemical proximate the surface of the body structure; and at least one computing device operably coupled to one or more of the plurality of spaced-apart independently actuatable reservoirs 110 and/or one or more resealable reservoirs 190 and configured to actuate one or more of the plurality of spaced-apart independently actuatable reservoirs between a reservoir discharge state and a reservoir retention state based on a comparison of a detected biological cell or biochemical to stored reference data. In an embodiment, the reservoirs are pre-loaded prior to implanting into a biological subject. In an embodiment, the reservoirs are refilled or loaded subsequent to implanting into a biological subject.

In an embodiment, one or more independently manipulatable immune cell stimulus delivering substrates 120 and/or one or more actuatable reservoirs 110 and/or one or more resealable reservoirs 190 includes at least one immune cell stimuli composition having one or more immune cell stimuli. Non-limiting examples of immune cell stimuli include adjuvants, allergens, anti-inflammatory agents, protease inhibitors or enzyme inhibitors, receptor agonists, receptor antagonists, therapeutic agents, tolerogens, toll-like receptor agonists, toll-like receptor antagonists, vaccines, or combinations thereof. In an embodiment, the microfluidic device includes at least one reservoir containing at least one antimicrobial agent.

Some non-limiting examples of antimicrobial agents include one or more pore-forming antimicrobial peptides. Antimicrobial peptides represent an abundant and diverse group of molecules that are naturally produced by many tissues and cell types in a variety of invertebrate, plant and animal species. The amino acid composition, amphipathicity, cationic charge and size of antimicrobial peptides allow them to attach to and insert into microbial membrane bilayers to form pores leading to cellular disruption and death. More than 800 different antimicrobial peptides have been identified or predicted from nucleic acid sequences, a subset of which are available in a public database (see, e.g., Wang & Wang, Nucleic Acids Res. 32:D169-D592, 2004); http://aps.unmc.edu/AP/main.php, which is incorporated herein by reference).

More specific examples of antimicrobial peptides include, among others, anionic peptides, e.g., maximin H5 from amphibians, small anionic peptides rich in glutamic and aspartic acids from sheep, cattle and humans, and dermcidin from humans; linear cationic alpha-helical peptides, e.g., cecropins (A), andropin, moricin, ceratotoxin, and melittin from insects, cecropin P1 from Ascaris nematodes, magainin 2, dermaseptin, bombinin, brevinin-1, esculentins and buforin II from amphibians, pleurocidin from skin mucous secretions of the winter flounder, seminalplasmin, BMAP, SMAP (SMAP29, ovispirin), PMAP from cattle, sheep and pigs, CAP18 from rabbits and LL37 from humans; cationic peptides enriched for specific amino acids, e.g., praline-containing peptides including abaecin from honeybees, praline- and arginine-containing peptides including apidaecins from honeybees, drosocin from Drosophila, pyrrhocoricin from European sap-sucking bug, bactenicins from cattle (Bac7), sheep and goats and PR-39 from pigs, praline- and phenylalanine-containing peptides including prophenin from pigs, glycine-containing peptides including hymenoptaecin from honeybees, glycine- and praline-containing peptides including coleoptericin and holotricin from beetles, tryptophan-containing peptides including indolicidin from cattle, and small histidine-rich salivary polypeptides, including histatins from humans and higher primates; anionic and cationic peptides that contain cysteine and from disulfide bonds, e.g., peptides with one disulphide bond including brevinins, peptides with two disulfide bonds including alpha-defensins from humans (HNP-1, HNP-2, cryptidins), rabbits (NP-1) and rats, beta-defensins from humans (HBD1, DEFB118), cattle, mice, rats, pigs, goats and poultry, and rhesus theta-defensin (RTD-1) from rhesus monkey, insect defensins (defensin A); and anionic and cationic peptide fragments of larger proteins, e.g., lactoferricin from lactoferrin, casocidin 1 from human casein, and antimicrobial domains from bovine alpha-lactalbumin, human hemoglobin, lysozyme, and ovalbumin (see, e.g., Brogden, Nat. Rev. Microbiol. 3:238-250, 2005, which is incorporated herein by reference).

Further non-limiting examples of antimicrobial agents include antibacterial drugs. Non-limiting examples of antibacterial drugs include beta-lactam compounds such as penicillin, methicillin, nafcillin, oxacillin, cloxacillin, dicloxacillin, ampicillin, ticarcillin, amoxicillin, carbenicillin, and piperacillin; cephalosporins and cephamycins such as cefadroxil, cefazolin, cephalexin, cephalothin, cephapirin, cephradine, cefaclor, cefamandole, cefonicid, cefuroxime, cefprozil, loracarbef, ceforanide, cefoxitin, cefmetazole, cefotetan, cefoperazone, cefotaxime, ceftazidine, ceftizoxine, ceftriaxone, cefixime, cefpodoxime, proxetil, cefdinir, cefditoren, pivoxil, ceftibuten, moxalactam, and cefepime; other beta-lactam drugs such as aztreonam, clavulanic acid, sulbactam, tazobactam, ertapenem, imipenem, and meropenem; other cell wall membrane immune cell stimuli such as vancomycin, teicoplanin, daptomycin, fosfomycin, bacitracin, and cycloserine; tetracyclines such as tetracycline, chlortetracycline, oxytetracycline, demeclocycline, methacycline, doxycycline, minocycline, and tigecycline; macrolides such as erythromycin, clarithromycin, azithromycin, and telithromycin; aminoglycosides such as streptomycin, neomycin, kanamycin, amikacin, gentamicin, tobramycin, sisomicin, and netilmicin; sulfonamides such as sulfacytine, sulfisoxazole, silfamethizole, sulfadiazine, sulfamethoxazole, sulfapyridine, and sulfadoxine; fluoroquinolones such as ciprofloxacin, gatifloxacin, gemifloxacin, levofloxacin, lomefloxacin, moxifloxacin, norfloxacin, and ofloxacin; antimycobacteria drugs such as isoniazid, rifampin, rifabutin, rifapentine, pyrazinamide, ethambutol, ethionamide, capreomycin, clofazimine, and dapsone; and miscellaneous antimicrobials such as colistimethate sodium, methenamine hippurate, methenamine mandelate, metronidazole, mupirocin, nitrofurantoin, polymyxin B, clindamycin, choramphenicol, quinupristin-dalfopristin, linezolid, spectrinomycin, trimethoprim, pyrimethamine, and trimethoprim-sulfamethoxazole.

Further non-limiting examples of antimicrobial agents include antifungal agents. Non-limiting examples of antifungal agents include anidulafungin, amphotericin B, butaconazole, butenafine, caspofungin, clotrimazole, econazole, fluconazole, flucytosine griseofulvin, itraconazole, ketoconazole, miconazole, micafungin, naftifine, natamycin, nystatin, oxiconazole, sulconazole, terbinafine, terconazole, tioconazole, tolnaftate, and/or voriconazole.

Further non-limiting examples of antimicrobial agents include anti-parasite agents. Non-limiting examples of anti-parasite agents include antimalaria drugs such as chloroquine, amodiaquine, quinine, quinidine, mefloquine, primaquine, sulfadoxine-pyrimethamine, atovaquone-proguanil, chlorproguanil-dapsone, proguanil, doxycycline, halofantrine, lumefantrine, and artemisinins; treatments for amebiasis such as metronidazole, iodoquinol, paromomycin, diloxanide furoate, pentamidine, sodium stibogluconate, emetine, and dehydroemetine; and other anti-parasite agents such as pentamidine, nitazoxanide, suramin, melarsoprol, eflornithine, nifurtimox, clindamycin, albendazole, and tinidazole. Further non-limiting examples of immune cell stimuli include ionic silver, (SilvaSorb®, Medline Industries, Inc), anti-microbial silver compositions (Arglaes®, Medline Industries, Inc), or the like. Further non-limiting examples of immune cell stimuli include superoxide-forming compositions. Further non-limiting examples of immune cell stimuli include oxazolidinones, gram-positive antibacterial agents, or the like. See, e.g., U.S. Pat. No. 7,322,965 (issued Jan. 29, 2008), which is incorporated herein by reference.

In an embodiment, the antimicrobial agent is an antimicrobial peptide. Amino acid sequence information for a subset of these can be found as part of a public database (see, e.g., Wang & Wang, Nucleic Acids Res. 32:D169-D592, 2004); http://aps.unmc.edu/AP/main.php, which is incorporated herein by reference). Alternatively, a phage library of random peptides can be used to screen for peptides with antimicrobial properties against live bacteria, fungi and/or parasites. The DNA sequence corresponding to an antimicrobial peptide can be generated ex vivo using standard recombinant DNA and protein purification techniques.

In an embodiment, the microfluidic device 102 includes one or more independently manipulatable immune cell stimulus delivering substrates or actuatable reservoirs are configured to deliver at least one immune cell stimuli from the one or more independently manipulatable immune cell stimulus delivering substrates 120 and/or one or more actuatable reservoirs 110 and/or one or more resealable reservoirs 190 to at least one of a region proximate an outer and an inner surface of the microfluidic device 102. In an embodiment, at least one of the one or more independently manipulatable immune cell stimulus delivering substrates 120 and/or one or more actuatable reservoirs 110 and/or one or more resealable reservoirs 190 are configured to deliver one or more immune cell stimuli in a spatially patterned distribution. In an embodiment, at least one of the one or more independently manipulatable immune cell stimulus delivering substrates 120 and/or one or more actuatable reservoirs 110 and/or one or more resealable reservoirs 190 are configured to deliver one or more immune cell stimuli in a temporally patterned distribution.

In an embodiment, the microfluidic device 102 includes at least one computing device 130 operably coupled to one or more of the plurality of spaced-apart independently manipulatable immune cell stimulus delivering substrates 120 and/or one or more actuatable reservoirs 110 and/or one or more resealable reservoirs 190 and configured to actuate one or more of the plurality of spaced-apart one or more independently manipulatable immune cell stimulus delivering substrates 120 and/or one or more actuatable reservoirs 110 and/or one or more resealable reservoirs 190 between an immune cell stimulus discharge state and an immune cell stimulus retention state. See FIG. 3 for details. In an embodiment, a computing device 130 is operable to actuate one or more of the plurality of spaced-apart one or more independently manipulatable immune cell stimulus delivering substrates 120 and/or one or more actuatable reservoirs 110 and/or one or more resealable reservoirs 190 between an immune cell stimulus discharge state and an immune cell stimulus retention state based on a comparison of a detected characteristic to stored reference data.

In an embodiment, the computing device 130 is operably coupled to the one or more independently manipulatable immune cell stimulus delivering substrates 120 and/or one or more actuatable reservoirs 110 and/or one or more resealable reservoirs 190 and configured to actively control one or more of the plurality of independently manipulatable immune cell stimulus delivering substrates or actuatable reservoirs. In an embodiment, at least one computing device 130 is operably coupled to one or more independently manipulatable immune cell stimulus delivering substrates 120 and/or one or more actuatable reservoirs 110 and/or one or more resealable reservoirs 190 and configured to control at least one of a release rate, a release amount, and a release pattern associated with a delivery of the one or more immune cell stimuli. In an embodiment, at least one processor 132 is operably coupled to one or more independently manipulatable immune cell stimulus delivering substrates 120 and/or one or more actuatable reservoirs 110 and/or one or more resealable reservoirs 190 and configured to control at least one of a release rate, a release amount, or a release pattern associated with the delivery of the one or more immune cell stimuli from the at least one independently manipulatable immune cell stimulus delivering substrates or actuatable reservoir proximate to a surface of the microfluidic device.

In an embodiment, a computing device 130 is operably coupled to the one or more independently manipulatable immune cell stimulus delivering substrates 120 and/or one or more actuatable reservoirs 110 and/or one or more resealable reservoirs 190 and configured to control at least one of an immune cell stimulus delivery rate, an immune cell stimulus delivery amount, an immune cell stimulus delivery composition, a port release rate, a port release amount, or a port release pattern.

In an embodiment, at least one computing device 130 is operably coupled to one or more of the plurality of spaced-apart one or more independently manipulatable immune cell stimulus delivering substrates 120 and/or one or more actuatable reservoirs 110 and/or one or more resealable reservoirs 190 and configured to actuate one or more of the plurality of spaced-apart one or more independently manipulatable immune cell stimulus delivering substrates 120 and/or one or more actuatable reservoirs 110 and/or one or more resealable reservoirs 190 between an immune cell stimulus discharge state and an immune cell stimulus retention state.

In an embodiment, the one or more reservoirs or substrates of the device include a series of different antigens or a series of different concentrations of the same antigen, thus the level or type of antigen released to the biological tissue can be controlled, thus actively controlling the immune response thereto. For example, in an early response to disease, presentation of high levels of CpG and GM-CSF in order to recruit antigen presenting cells to the device, differentiate them, and/or activate them. Such presentation, for example, will drive increased production of CD8+ T cells for destruction of diseased cells, and control of disease. Subsequently, presentation of CpG will be reduced or eliminated, and GM-CSF presentation will continue, assisting in driving a T regulatory and T helper cell population as the cytolytic T cell events subside. In an embodiment, a system or device disclosed herein includes regulation of the type of antigen presenting cell recruited and/or activated. For example, lymphoid or plasmacytoid dendritic cells (pDCs) and “conventional” or myeloid dendritic cells (mDCs) express different receptors on their cell surfaces, arise from different precursors (lymphoid vs. myeloid, respectively) and function differently during infection or disease state, and pre-pDCs are precursors to pDCs with characteristics different from either the pDCs or mDCs. For example, while all of the dendritic cells recognized to date share antigen presenting cell function, the mDCs are segregated into subsets (such as Langerhans cells, interstitial dendritic cells, dermal dendritic cells, etc.), while the pDCs have not. Further, pDCs generally mature and secrete large amounts of interferons alpha and beta, usually express CD4 and MHC II, but not T cell or B cell lineage markers. See for example, McKenna, et al. J. Virol. 2005, 79(1):17, which is incorporated herein by reference. For example, once dendritic cells mature (e.g., by way of a pattern recognition receptor such as a toll-receptor), the cells phagocytose pathogens, break them down, and degrade the corresponding proteins—presenting fragments of these proteins on their cell surface using MHC Class I, II, or III. The mature dendritic cell upregulates particular cell surface receptors, such as CD80, CD86, and CD40, as well as chemotactic receptors such as CCR7, which assists in migration of the dendritic cell through the subject's body to a lymph node or spleen.

For example, CpG oligonucleotides are isolated from endogenous sources or synthesized in vivo or in vitro, and include, for example, exogenous microorganisms, fungi, bacteria, protozoa, viruses, or other parasites, as well as endogenous sources such as benign or malignant neoplastic tumors. CpG can be synthesized, for example, by utilizing PCR or other adapted molecular biology techniques.

Likewise, in an embodiment, once the antigen presenting cells or other biological cells are no longer desired near the device for activation, the device repels the cells in a manner sufficient to drive the immune response (e.g., to reduce a heightened immune response). Thus, in an embodiment, the device further includes a selectively activatable repelling mechanism. In an embodiment, the at least one selectively activatable repelling mechanism is configured to repel at least one biological cell from the device. In an embodiment, the at least one selectively activatable repelling mechanism is configured to repel at least one immune cell from the device. In an embodiment, the at least one selectively activatable repelling mechanism is configured to repel at least one immune cell from the device subsequent to actuation of at least one of the actuatable reservoirs. In an embodiment, the at least one selectively and activatable repelling mechanism is configured to repel at least one biological cell from the tissue in which the device is implanted. In an embodiment, the at least one selectively and activatable repelling mechanism includes one or more of a chemical, electrical, magnetic, electromagnetic, thermal, or other means. In an embodiment, the at least one selectively and activatable repelling mechanism includes one or more reservoirs containing at least one biochemical sufficient to repel an immune cell. In an embodiment, the at least one selectively and activatable repelling mechanism includes at least one electroactive polymer, thermal-active polymer, magnetic-active polymer, or light-active polymer.

As disclosed herein, the repelling mechanism includes chemical, electrical, magnetic, thermal, electromagnetic, or other means. For example, a wireless induction heating system can be utilized that includes thermal responses of materials (e.g., nickel, iron, copper, etc.) in heating units through application of an alternating magnetic field. See, for example, Baek, et al., Lab Chip 7:10; pp. 909-17 (2010) (Abstract), which is incorporated herein by reference. In an embodiment, the heating units are controlled by remote control. In an embodiment, the remote control is wireless. In an embodiment, the device further includes a thermostat or rheostat configured to regulate the one or more heating units.

In an embodiment, the immune cell stimulus includes an immune cell activator. In an embodiment, the immune cell stimulus includes an immune cell repressor. In an embodiment, the immune cell stimulus at least one of recruits or activates one or more antigen presenting cells. In an embodiment, the immune cell stimulus includes one or more cell regulatory molecules (e.g., immune cell regulatory molecules).

As described herein elsewhere, in an embodiment, the immune cell regulatory molecule includes at least one of cytokine, a chemokine, an antigen, a vaccine, an adjuvant, or a co-stimulatory molecule. In an embodiment, the immune cell regulatory molecule includes at least one of CpG oligonucleotides, chemokine, ICAM, anti CTLA-4 antibodies, TGF-beta, LPS, Fas ligand, TRAIL, lymphotactin, M-FP, heat shock proteins, CD3, CD28, CD80, CD86, ICOS-L, ICOS, CD40, CD40L, CD154, CD19, CD81, CD21, iC3b, C3dg, C3d, a caspase, granzyme B, FasL, Fas receptor, TRAIL receptor, apoptosome, integrin, laminin, elastin, fibrin, fibrinogen, collagen, fibronectin, TNF receptor, bcl-2, GM-CSF, G-CSF, IL-1, IL-2, IL-3, IL-4, IL-5, IL-6, IL-8, IL-10, IL-12, IL-15, IL-17, IL-18, CCL19, TNF alpha, IFN-alpha, Flt-3 ligand, CCL21, M-CSF, MIF, or IFN-gamma.

In an embodiment, the caspase includes one or more of CASP1, CASP2, CASP3, CASP4, CASP5, CASP6, CASP7, CASP8, CASP9, CASP10, CASP11, CASP12, CASP13, CASP14, CASP15, CASP16, CASP17, or the like.

Non-limiting examples of α-Globulins examples include α1-acid glycoprotein, α1-antichymotrypsin, α1-antitrypsin, α1B-glycoprotein, α1-fetoprotein, α1-microglobulin, α1T-glycoprotein, α2HS-glycoprotein, α2-macroglobulin, 3.1 S Leucine-rich α2-glycoprotein, 3.8 S histidine-rich α2-glycoprotein, 4 S α2, α1-glycoprotein, 8 S α3-glycoprotein, 9.5 S α1-glycoprotein (serum amyloid P protein), Corticosteroid-binding globulin, ceruloplasmin, GC globulin, haptoglobin (e.g., Type 1-1, Type 2-1, or Type 2-2), inter-α-trypsin inhibitor, pregnancy-associated α2-glycoprotein, serum cholinesterase, thyroxine-binding globulin, transcortin, vitamin D-binding protein, Zn-α2-glycoprotein, or the like. Among β-globulins, examples include, but are not limited to, hemopexin, transferrin, β2-microglobulin, β2-glycoprotein I, β2-glycoprotein II, (C3 proactivator), β2-glycoprotein III, C-reactive protein, fibronectin, pregnancy-specific β1-glycoprotein, ovotransferrin, or the like. Among immunoglobulins examples include, but are not limited to, immunoglobulin G (e.g., IgG, IgG₁, IgG₂, IgG₃, IgG₄), immunoglobulin A (e.g., IgA, IgA₁, IgA₂), immunoglobulin M, immunoglobulin D, immunoglobulin E, κ Bence Jones protein, γ Bence Jones protein, J Chain, or the like.

Among apolipoproteins examples include, but are not limited to, apolipoprotein A-I (HDL), apolipoprotein A-II (HDL), apolipoprotein C-I (VLDL), apolipoprotein C-II, apolipoprotein C-III (VLDL), apolipoprotein E, or the like. Among γ-mobility (non-immunoglobulin) examples include, but are not limited to, 0.6 S γ2-globulin, 2 S γ2-globulin, basic Protein B2, post-γ-globulin (γ-trace), or the like. Among low-molecular weight proteins examples include, but are not limited to, lysozyme, basic protein B1, basic protein B2, 0.6 S γ2-globulin, 2 S γ 2-globulin, post γ-globulin, or the like.

Among complement components examples include, but are not limited to, C1 esterase inhibitor, C1q component, C1r component, C1s component, C2 component, C3 component, C3a component, C3b-inactivator, C4 binding protein, C4 component, C4a component, C4-binding protein, C5 component, C5a component, C6 component, C7 component, C8 component, C9 component, factor B, factor B (C3 proactivator), factor D, factor D (C3 proactivator convertase), factor H, factor H (β₁H), properdin, or the like. Among coagulation proteins examples include, but are not limited to, antithrombin III, prothrombin, antihemophilic factor (factor VIII), plasminogen, fibrin-stabilizing factor (factor XIII), fibrinogen, thrombin, or the like.

Among cell-Related Plasma Proteins examples include, but are not limited to, fibronectin, β-thromboglobulin, platelet factor-4, serum Basic Protease Inhibitor, or the like. Among amyloid proteins (Non-Immunoglobulin) examples include, but are not limited to, amyloid-Related apoprotein (apoSAA1), AA (FMF) (ASF), AA (TH) (AS), serum amyloid P component (9.5 S 7α1-glycoprotein), or the like. Among miscellaneous trace components examples include, but are not limited to, varcinoembryonic antigen, angiotensinogen, or the like.

In an embodiment, the system 100 includes one or more sensors 165. In an embodiment, the microfluidic device 102 includes one or more of the sensors 165. In an embodiment, the sensor component 160 includes one or more sensors 165.

Non-limiting examples of sensors 165 include acoustic wave sensors, aptamer-based sensors, biosensors, blood volume pulse sensors, cantilevers, conductance sensors, electrochemical sensors, fluorescence sensors, force sensors, heat sensors (e.g., thermistors, thermocouples, or the like), high resolution temperature sensors, differential calorimeter sensors, optical sensors, goniometry sensors, potentiometer sensors, resistance sensors, respiration sensors, sound sensors (e.g., ultrasound), Surface Plasmon Band Gap sensor (SPRBG), physiological sensors, surface plasmon sensors, or the like. Further non-limiting examples of sensors 165 include affinity sensors, bioprobes, biostatistics sensors, enzymatic sensors, in-situ sensors (e.g., in-situ chemical sensor), ion sensors, light sensors (e.g., visible, infrared, or the like), microbiological sensors, microhotplate sensors, micron-scale moisture sensors, nanosensors, optical chemical sensors, single particle sensors, or the like.

Further non-limiting examples of sensors 165 include chemical sensors, cavitand-based supramolecular sensors, nucleic acid sensors, deoxyribonucleic acid sensors (e.g., electrochemical DNA sensors, or the like), supramolecular sensors, or the like. In an embodiment, at least one of the one or more sensors 165 is configured to detect or measure the presence or concentration of specific target chemicals (e.g., blood components, biological sample component, cerebral spinal fluid component, lymph components, interstitial fluid components, infectious agents, infection indication chemicals, inflammation indication chemicals, diseased tissue indication chemicals, biological agents, molecules, ions, or the like).

Further non-limiting examples of sensors 165 include chemical transducers, ion sensitive field effect transistors (ISFETs), ISFET pH sensors, membrane-ISFET devices (MEMFET), microelectronic ion-sensitive devices, potentiometric ion sensors, quadruple-function ChemFET (chemical-sensitive field-effect transistor) integrated-circuit sensors, sensors with ion-sensitivity and selectivity to different ionic species, or the like. Further non-limiting examples of the one or more sensors 165 can be found in the following documents: U.S. Pat. Nos. 7,396,676, and 6,831,748; each of which is incorporated herein by reference.

In an embodiment, the one or more sensors 165 include one or more acoustic transducers, electrochemical transducers, photochemical transducer, optical transducers, piezoelectrical transducers, or thermal transducers. For example, in an embodiment, the one or more sensors 165 include one or more acoustic transducers. In an embodiment, the one or more sensors 165 include one or more thermal detectors, photovoltaic detectors, or photomultiplier detectors. In an embodiment, the one or more sensors 165 include one or more charge coupled devices, complementary metal-oxide-semiconductor devices, photodiode image sensor devices, whispering gallery mode micro cavity devices, or scintillation detector devices. In an embodiment, the one or more sensors 165 include one or more complementary metal-oxide-semiconductor image sensors.

In an embodiment, the one or more sensors 165 include one or more conductivity sensor. In an embodiment, the one or more sensors 165 include one or more spectrometers. In an embodiment, the one or more sensors include one or more Bayer sensors. In an embodiment, the one or more sensors include one or more Foveon sensors. In an embodiment, the one or more sensors 165 include one or more density sensors. In an embodiment, the one or more density sensors include one or more optical density sensors. In an embodiment, the one or more density sensors include one or more refractive index sensors. In an embodiment, the one or more refractive index sensors include one or more fiber optic refractive index sensors.

In an embodiment, the one or more sensors 165 include one or more surface plasmon resonance sensors. In an embodiment, the one or more sensors 165 are configured to detect target molecules, such as biochemicals or molecules from biological cells. For example, surface-plasmon-resonance-based-sensors detect target molecules suspended in a fluid, for example, by reflecting light off thin metal films in contact with the fluid. Adsorbing molecules cause changes in the local index of refraction, resulting in detectable changes in the resonance conditions of the surface plasmon waves.

In an embodiment, the one or more sensors 165 include one or more localized surface plasmon resonance sensors. In an embodiment, detection of target molecules includes monitoring shifts in the resonance conditions of the surface plasmon waves due to changes in the local index of refraction associates with adsorption of target molecules. In an embodiment, the one or more sensors 165 include one or more functionalized cantilevers. In an embodiment, the one or more sensors 165 include a light transmissive support and a reflective metal layer.

In an embodiment, the one or more sensors 165 include one or more acoustic biosensors, amperometric biosensors, calorimetric biosensors, optical biosensors, or potentiometric biosensors. In an embodiment, the one or more sensors 165 include one or more fluid flow sensors. In an embodiment, the one or more sensors 165 include one or more differential electrodes. In an embodiment, the one or more sensors 165 include one or more biomass sensors. In an embodiment, the one or more sensors 165 include one or more immunosensors.

In an embodiment, one or more of the sensors 165 are configured to detect at least one characteristic associated with a biological subject. In an embodiment, one or more of the sensors 165 are configured to detect at least one characteristic associated with a biological sample (e.g., tissue, biological fluid, target sample, or the like). For example, in an embodiment, at least one of the one or more sensors 165 is configured to detect at least one characteristic associated with a biological sample proximate a surface (e.g., outer surface 108 or inner surface 110, or the like) of the microfluidic device 102. In an embodiment, one or more of the sensors 165 are configured to detect at least one of a characteristic of a biological sample proximate the microfluidic device 102, a characteristic of a tissue proximate the microfluidic device 102, and a physiological characteristic of the biological subject. In an embodiment, one or more of the sensors 165 are configured to determine one or more tissue spectroscopic properties, such as, for example, a transport scattering coefficient, an extinction coefficient, an absorption coefficient, a remittance, a transmittance, or the like.

In an embodiment, the at least one characteristic includes a physiological characteristic of the biological subject. Physiological characteristics such as, for example pH can be used to assess blood flow, a cell metabolic state (e.g., anaerobic metabolism, or the like), the presence of an infectious agent, a disease state, or the like. Among physiological characteristics examples include, but are not limited to, at least one of a temperature, a regional or local temperature, a pH, an impedance, a density, a sodium ion level, a calcium ion level, a potassium ion level, a glucose level, a lipoprotein level, a cholesterol level, a triglyceride level, a hormone level, a blood oxygen level, a pulse rate, a blood pressure, a respiratory rate, a vital statistic, a cell surface molecule, a secreted cell molecule, a biochemical, an immune regulatory molecule, or the like.

In an embodiment, an immune regulatory molecule is contained within a reservoir or substrate of the device. In an embodiment, the immune cell regulatory molecule includes at least one of CpG oligonucleotides, chemokine, ICAM, anti CTLA-4 antibodies, TGF-beta, LPS, Fas ligand, TRAIL, lymphotactin, M-FP, heat shock proteins, CD3, CD28, CD80, CD86, ICOS-L, ICOS, CD40, CD40L, CD154, CD19, CD81, CD21, iC3b, C3dg, C3d, a caspase, granzyme B, FasL, Fas receptor, TRAIL receptor, apoptosome, integrin, laminin, elastin, fibrin, fibrinogen, collagen, fibronectin, TNF receptor, bcl-2, GM-CSF, G-CSF, IL-1, IL-2, IL-3, IL-4, IL-5, IL-6, IL-8, IL-10, IL-12, IL-15, IL-17, IL-18, CCL19, TNF alpha, IFN-alpha, Flt-3 ligand, CCL21, M-CSF, MIF, or IFN-gamma.

In an embodiment, the caspase includes one or more of CASP1, CASP2, CASP3, CASP4, CASP5, CASP6, CASP7, CASP8, CASP9, CASP10, CASP11, CASP12, CASP13, CASP14, CASP15, CASP16, CASP17, or the like.

In an embodiment, the at least one characteristic includes one or more parameters associated with at least one of leukopenia, leukophilia, lymphocytopenia, lymphocytophilia, neutropenia, neutrophilia, thrombocytopenia, disseminated intravascular coagulation, bacteremia, and viremia. In an embodiment, the at least one characteristic includes at least one of an infection marker, an inflammation marker, an infective stress marker, a systemic inflammatory response syndrome marker, or a sepsis marker. In an embodiment, the infection marker includes at least one of a red blood cell count, a lymphocyte level, an antigen presenting cell level, a cell surface marker, a leukocyte count, a myeloid count, an erythrocyte sedimentation rate, or a C-reactive protein level. In an embodiment, the at least one characteristic includes at least one of a cytokine plasma concentration or an acute phase protein plasma concentration.

In an embodiment, the system 100 includes one or more computing devices 130 operably coupled to one or more sensors 165. In an embodiment, at least one computing device 130 is configured to process an output associated with one or more sensors 165. In an embodiment, the system 100 includes one or more computing devices 130 configured to concurrently or sequentially operate multiple sensors 165. In an embodiment, the system 100 is configured to compare an input associated with at least one characteristic associated with a tissue proximate a microfluidic device 102 to a data structure 260 including reference values, and to generate a response based in part on the comparison. In an embodiment, the system 100 is configured to compare an input associated with at least one physiological characteristic associated with a biological subject to a data structure 260 including reference values, and to generate a response based in part on the comparison. In an embodiment, the system 100 is configured to compare an input associated with at least one characteristic associated with a tissue proximate a microfluidic device 102 to a data structure 260 including reference values, and to generate a response based in part on the comparison.

In an embodiment, at least one computing device 130 is configured to perform a comparison of at least one detected characteristic to stored reference data, and to generate a response based at least in part on the comparison. For example, in an embodiment, at least one computing device 130 is configured to perform a comparison of at least one characteristic associated with the biological sample to stored reference data, and to initiate a treatment protocol based at least in part on the comparison. In an embodiment, one or more computing devices 130 are communicatively coupled to one or more sensors 165 and configured to actuate a determination of the at least one characteristic associated with a biological tissue proximate a surface of the microfluidic device 102.

In an embodiment, the system 100 includes, among other things, circuitry 167 configured to determine a sensed event, such as the presence of a biochemical or biological cell, in one or more regions in the vicinity of the microfluidic device 102, for example, proximate the surface of the body structure 104 of the device. In an embodiment, circuitry includes one or more components operably coupled (e.g., communicatively coupled, electromagnetically, magnetically, acoustically, optically, inductively, electrically, capacitively coupleable, or the like) to each other. In an embodiment, circuitry includes one or more remotely located components. In an embodiment, remotely located components are operably coupled via wireless communication. In an embodiment, remotely located components are operably coupled via one or more receivers, transmitters, transceivers, or the like.

In an embodiment, the circuitry 167 configured to determine the sensed event, such as the presence of a biochemical or biological cell, includes at least one sensor component 160 having one or more sensors 165. In an embodiment, the circuitry 167 configured to determine the sensed event such as the presence of a biochemical or biological cell includes at least one sensor component 160 having a component identification code and configured to implement instructions addressed to the sensor component 160 according to the component identification code. In an embodiment, the circuitry 167 configured to determine the presence of an immune cell or biochemical includes at least one sensor component 160 operably coupled to a biochemical or biological cell component or marker array.

In an embodiment, the circuitry 167 configured to determine the presence of a biological cell or biochemical includes at least one of a charge-coupled device, a complementary metal-oxide-semiconductor device, a photodiode image sensor device, a Whispering Gallery Mode (WGM) micro cavity device, or a scintillation detector device. In an embodiment, the circuitry 167 configured to determine the presence of a biological cell or biochemical includes at least one photoelectric device.

In an embodiment, the circuitry 167 configured to determine the presence of a particular biochemical or biological cell (e.g., an immune cell such as an antigen presenting cell) includes a wavelength-tunable surface plasmon resonance sensor. In an embodiment, the circuitry 167 configured to determine the presence of an immune cell or biochemical includes a surface plasmon resonance microarray sensor having a wavelength-tunable metal-coated grating. In an embodiment, the circuitry 167 configured to determine the biochemical or immune cell presence includes one or more acoustic transducers, electrochemical transducers, optical transducers, piezoelectric transducers, or thermal transducers. In an embodiment, the circuitry 167 configured to determine the presence of an immune cell or biochemical includes one or more thermal detectors, photovoltaic detectors, or photomultiplier detectors. In an embodiment, the circuitry 167 configured to determine the immune cell or biochemical includes one or more charge-coupled devices, complementary metal-oxide-semiconductor devices, photodiode image sensor devices, whispering gallery mode micro cavity devices, or scintillation detector devices. In an embodiment, the circuitry 167 configured to determine the biochemical or immune cell includes one or more acoustic transducers. In an embodiment, the circuitry 167 configured to determine the immune cell or biochemical includes one or more density sensors. In an embodiment, the circuitry 167 configured to determine the immune cell or biochemical includes one or more optical density sensors. In an embodiment, the circuitry 167 configured to determine the biochemical or immune cell includes one or more photoacoustic spectrometers. As disclosed in other areas, in an embodiment, one or more first sensed events (e.g., presence of a biological cell or biochemical) is stored. In an embodiment, a second sensed event is compared with the first sensed event, and the difference can be determined therefrom. Thus, in an embodiment, the device senses the presence of a biological cell or biochemical, for example, and correspondingly senses the absence thereof through a comparison of sensed events. For example, the one or more sensors are configured to sense the presence of a biological cell or biochemical and in future sensed events circuitry is configured to compare the presence or absence of the biological cell or biochemical over time. In an embodiment, the comparison occurs in real-time as described herein at other areas.

In an embodiment, the circuitry 167 configured to determine the presence of a biochemical or immune cell includes one or more refractive index sensors. In an embodiment, the circuitry 167 configured to determine the biochemical or immune cell includes one or more fiber optic refractive index sensors. In an embodiment, the circuitry 167 configured to determine the biochemical or immune cell includes one or more surface plasmon resonance sensors. In an embodiment, the circuitry 167 configured to determine the biochemical or immune cell includes one or more localized surface plasmon resonance sensors.

In an embodiment, the circuitry 167 configured to determine the presence of a biochemical or biological cell includes a light transmissive support and a reflective metal layer. In an embodiment, the circuitry 167 configured to determine the presence of a biochemical or biological cell includes one or more acoustic biosensors, amperometric biosensors, calorimetric biosensors, optical biosensors, or potentiometric biosensors. In an embodiment, the circuitry 167 configured to determine the presence of a biochemical or biological cell includes one or more differential electrodes.

In an embodiment, the circuitry 167 configured to determine the presence of a biochemical or biological cell includes one or more biomass sensors, immunosensors, or functionalized cantilevers.

In an embodiment, the system 100 includes, among other things, circuitry 156 configured to obtain information. In an embodiment, the circuitry 156 configured to obtain information includes circuitry 156 configured to obtain information associated with a delivery of the immune cell stimulus. In an embodiment, the circuitry 156 configured to obtain information includes circuitry configured to obtain at least one of a command stream, a software stream, or a data stream.

In an embodiment, the system 100 includes, among other things, circuitry 157 configured to store information. In an embodiment, the circuitry 157 configured to store information includes one or more data structures.

In an embodiment, the system 100 includes, among other things, circuitry 163 configured to provide information. In an embodiment, the circuitry 163 configured to provide information includes circuitry 163 configured to provide information related to one or more biochemicals, one or more biological cells, or status update information related to the microfluidic device.

In an embodiment, the system 100 includes, among other things, circuitry 169 configured to perform a comparison of the determined at least one characteristic associated with the tissue or a biological fluid proximate the microfluidic device 102 to stored reference data following the delivery of the immune cell stimulus. In an embodiment, the microfluidic device 102 includes, among other things, circuitry configured to generate a response based at least in part on the comparison. In an embodiment, the circuitry 169 configured to perform a comparison includes, among other things, one or computing devices 130 configured to perform a comparison of the at least one characteristic associated with the tissue or a biological fluid proximate the microfluidic device 102 stored reference data following delivery of the immune cell stimulus, and to generate a response based at least in part on the comparison. In an embodiment, the comparison includes one or more computing devices configured to perform a comparison of the temporal and/or spatial presence (or absence) of a selected biochemical (e.g., antigen or cytokine, etc.) or selected biological cell (e.g., immune cell). In an embodiment, the system 100 is configured to initiate one or more treatment protocols. For example, a treatment protocol can include a temporal and/or spatial program for delivering a particular antigen or cytokine expected to elicit a specific immune response. Further, for example, the treatment protocol can include a program for multiple antigens or cytokines to be delivered at particular temporal or spatial points.

Many of the disclosed embodiments can be electrical, electromechanical, software-implemented, firmware-implemented, or other otherwise implemented, or combinations thereof. Many of the disclosed embodiments can be software or otherwise in memory, such as one or more executable instruction sequences or supplemental information as described herein. For example, in an embodiment, in an embodiment, the microfluidic device 102 includes, among other things, one or more computing devices 130 configured to perform a comparison of the at least one characteristic associated with the biological subject to stored reference data, and to generate a response based at least in part on the comparison. In an embodiment, one or more computing devices 130 are configured to automatically control one or more of a frequency, duration, a pulse rate, a duty cycle, or the like associated with an acoustic energy generated by the one or more transducers 185 based on a sensed parameter. In an embodiment, one or more computing devices 130 are configured to automatically control one or more of a frequency, a duration, a pulse rate, a duty cycle, or the like associated with the acoustic energy generated by the one or more transducers 185 based on a sensed parameter associated with a region within the biological subject.

In an embodiment, one or more immune cell stimuli are carried by vesicles (e.g., ethasomes, hydrogels, liposomes, micelles, microspheres, niosomes, lipospheres, non-ionic surfactant vesicles, organogels, phospholipid surfactant vesicles, transfersomes, virosomes, or the like.).

In an embodiment, one or more immune cell stimuli are conjugated to or encapsulated in one or more remotely releasable delivery systems configured for release from a substrate or reservoir of the microfluidic device 102. In an embodiment, the releasable delivery system is designed for single release or for repeated release of one or more immune cell stimuli. In an embodiment, the triggered delivery system releases one or more immune cell stimuli in response to temperature, electromagnetic radiation (e.g., UV, visible or near infrared radiation, radiofrequency, microwave, or the like), a magnetic field, ultrasound, electric field, or the like. For example, in an embodiment, application of electromagnetic radiation, a magnetic field, ultrasound, or the like can induce a thermal change sufficient for release of one or more immune cell stimuli from a temperature-sensitive releasable delivery system.

In an embodiment, the releasable delivery system includes, among other things, liposomes, polymer vesicles, polymeric liposomes, polyelectrolyte microcontainers, multilayered capsules, micelles, dendrimers, microbubbles, or the like. In an embodiment, polymers are cross-linked with photolabile groups, allowing immune cell stimuli to be released in response to light. An example of a photocleavable molecule includes among other things 2-nitrobenzyl ester. In an embodiment, one or more immune cell stimuli are released from the delivery system by the reversible isomerization of molecules upon irradiation with near-UV or visible light. UV irradiation, for example, can induce phase transitions of natural and synthetic polymers, accompanied by reversible volume changes, allowing immune cell stimuli to be released as the polymers shrink or swell. For example, azobenzenes which contain two phenyl groups and undergo conformational changes in response to UV light can be used as part of a molecular valve to control release of one or more immune cell stimuli through a channel protein incorporated into liposomes. In an embodiment, at least a portion of the surface of the device includes a thermal-active, electroactive, magnetic active, or other responsive polymer. Thus, induction of the responsive polymer can cause release of the releasable delivery system. For example, in an embodiment, the body structure of the device includes at least one of an electroactive polymer, thermal-active polymer, magnetic-active polymer, or light-active polymer. In an embodiment, the surface of the body structure of the device (e.g., the remainder of the body structure can include some other material) includes at least one electroactive polymer, thermal-active polymer, magnetic-active polymer, or light-active polymer.

In an embodiment, for example, polymers are combined with magnetic oxide nanoparticles to form ferrogel materials which deform in response to a magnetic field, allowing for releasable release of one or more immune cell stimuli. In an embodiment, ferrogel materials include, among other things, ferrite particles cross-linked to or embedded in poly(vinyl alcohol), polyNIPAm, or gelatin. In the case of microbubbles, in an embodiment, ultrasound is used to trigger release of a gas from a stabilizing shell of lipid or polymer or, under conditions of low frequency, ultrasound can induce cavitation of microbubbles and disruption of nearby cell membranes sufficient to allow passage into the cells of co-administered immune cell stimuli. In an embodiment, magnetic nanoparticles (e.g. approximately 20-100 nm) are embedded in a flexible polymer (e.g., polydimethylsiloxane) for control of active delivery of the contents of the device.

In an embodiment, the releasable delivery system includes, among other things, metallic nanostructures, particularly gold nanostructures. In an embodiment, under optical irradiation, electrons associated with metallic nanostructures oscillate in phase, a phenomenon referred to as surface plasmon resonance. In their excited state, the electrons subsequently decay through either radiative (fluorescence), nonradiative (lattice rearrangement), or photothermal (local heating) pathways. The specific decay pathway is dependent on the geometry of the nanoparticles and the nature of the excitation pulse. In an embodiment, lattice rearrangement and local heating induced in this manner can be used to trigger delivery of immune cell stimuli. As a non-limiting example, gold nanorods can be melted into nanospheres using ultrafast laser pulses, effectively triggering release of surface-bound immune cell stimuli as the gold lattice atoms rearrange. Heterogeneous mixtures of rods or rodlike structures with distinct geometries and resonant frequencies enable selective release of multiple ligands. For example, gold nanocapsules and gold nanorods exhibit SPR longitudinal modes at 800 nm and 1100 nm, respectively. Pulsed laser irradiation centered at either of these two resonant frequencies yields selective melting of the corresponding nanoparticles and selective release of associated immune cell stimuli. Weakly bound ligands can also be released by localized heating below the nanoparticle melting threshold. Gold nanoparticles can also be configured into nanoshells (i.e., hollow or enclosed solid cores) or nanocages (i.e., hollow interior and porous walls).

In an embodiment, the releasable delivery system includes a combination of liposomes or polymers and gold nanoparticles. In an embodiment, gold nanoparticle are combined with temperature-sensitive polymers for triggered release with near infrared radiation. In an embodiment, one or more immune cell stimuli can be incorporated into gold cages covered with monolayers of heat labile polymer chains, formed by polymerizing polymers, e.g., n-isopropylacrylamine (NIPAm) and acrylamide (Am) precursors, with a disulfide initiator, the poly(NIPAm-co-Am) chains attached to the surface of the gold cages by Au—S linkages, forming a hydrophobic layer with lower critical solution temperatures tunable between about 32° C. to about 50° C. In another non-limiting example, one or more immune cell stimuli can be co-encapsulated in liposomes in the presence of gold nanoparticles, the latter of which, in the presence of near infrared radiation, generate heat sufficient to disrupt the liposomes.

In an embodiment, releasable membranes can be used as walls of reservoirs, allowing a large quantity of immune cell stimuli to be contained and repeatedly released over time. For example, nanocomposite membranes consisting of a thermosensitive material, e.g., polyNIPAm-based nanogels and magnetic particles embedded in an ethylcellulose matrix, can be designed to achieve on-demand drug delivery upon application of an AC magnetic field. Alternatively, one or more immune cell stimuli can be released from magnetically actuated microchips configured with an array of wells and a biodegradable covering such as, for example, poly-(D,L-lactic acid). In an embodiment, an immune cell stimuli can be electrodeposited onto a thin film in the presence of magnetic oxide, e.g., Fe₃O₄/SiO₂, and released in response to a magnetic field. For further examples of releasable delivery systems, see e.g., Timko et al., Remotely Releasable Drug Delivery Systems. Advanced Materials, n/a. doi: 10.1002/adma.201002072 (2010) (Abstract only, pp. 1-3); Tsutsui et al., The Use of Microbubbles to Target Drug Delivery, Cardiovascular Ultrasound (2004) (Abstract only, one page).

Also described herein include methods of using the devices, or systems disclosed. For example, in an embodiment, a method of regulating an immune cell response from an at least partially implanted microfluidic device comprises selectively and actively releasing one or more biochemicals or one or more biological cells to one or more regions proximate the surface of an implanted portion of the microfluidic device via one or more actuatable reservoirs, and delivering a patterned immune cell stimulus composition to the one or more regions proximate the surface of the implanted portion of the microfluidic device in response to an automatically detected parameter associated with a biological sample proximate the surface of the implanted portion of the microfluidic device via one or more sensors of the device.

In an embodiment, selectively and actively releasing the one or more biochemical or one or more biological cells includes delivering an immune cell stimulus to one or more regions proximate the surface of the implanted portion determined to have an immune cell present, the immune cell stimulus at a dose sufficient to modulate an activity of the present immune cell.

In an embodiment, selectively and actively releasing the one or more biochemical or one or more biological cells includes delivering at least one of an immune cell regulatory molecule, antigen, biological cell, detectable indicator, or surface antimicrobial agent in response to an automatically detected parameter associated with a biological sample proximate the surface of the implanted portion of the microfluidic device.

In an embodiment, selectively and actively releasing the one or more biochemical or one or more biological cells includes delivering at least a first immune cell stimulus and a second immune cell stimulus to the one or more regions, the first immune cell stimulus and the second immune cell stimulus being different stimuli.

In an embodiment, selectively and actively releasing the one or more biochemical or one or more biological cells includes concurrently or sequentially delivering at least a first immune cell stimulus to a first region and a second immune cell stimulus to a second region.

In an embodiment, selectively and actively releasing the one or more biochemical or one or more biological cells includes concurrently or sequentially delivering at least a first spatially patterned immune cell stimulus to a first region and a second spatially patterned immune cell stimulus to a second region.

In an embodiment, selectively and actively releasing the one or more biochemical or one or more biological cells includes delivering a temporally patterned immune cell stimulus to one or more regions of the implanted portion of the device.

In an embodiment, the method further includes detecting at least one biological cell or at least one biochemical proximate the surface of the body structure of the implanted portion of the microfluidic device.

In an embodiment, the method further includes receiving information based at least in part on whether a detected biological cell or detected biochemical proximate the surface of the body structure that satisfies a target condition.

In an embodiment, the target condition includes at least one of a type of biological cell, type of biochemical, timing of delivery of an immune cell stimulus, response based on the type of surface of the body structure of the device, or spatial delivery of an immune cell stimulus.

In an embodiment, the method further includes sending information based at least in part on a detected biological cell or biochemical proximate the surface of the body structure that satisfies a target condition.

In an embodiment, a method comprises concurrently or sequentially delivering to one or more regions proximate the surface of an implantable microfluidics device a spatially patterned immune cell stimulus via a plurality of independently manipulatable immune cell stimulus delivering substrates configured to independently activate in response to a real-time detected parameter associated with a biological sample within the one or more regions proximate the surface of the microfluidics device. In an embodiment, concurrently or sequentially delivering to one or more regions proximate the surface of the microfluidics device the spatially patterned immune cell stimulus includes delivering a temporally patterned polymer-responsive stimulus having at least a first in time pattern and a second in time pattern, the first and second patterns being different.

In an embodiment, a method comprises concurrently or sequentially delivering to one or more regions proximate the surface of the microfluidics device a temporally patterned immune cell stimulus via a plurality of independently manipulatable immune cell stimulus delivering substrates configured to independently activate in response to a real-time detected parameter associated with at least one of biochemical information or biological cell information associated with a biological sample within one or more regions proximate the surface of the microfluidics device.

Some non-limiting examples of particular embodiments have been described herein for the following prophetic examples.

### PROPHETIC EXAMPLES

### Prophetic Example 1

### An Implanted Device to Exert Active Control Over Immune Cells

An implant device with reservoirs and micropumps actively delivers chemicals and biomolecules to multiple chambers and conduits in the device to control immune cells. The implant device also has aptamer-based microfluidics to detect and control the flow of immune cells. The device has microcircuitry which responds to signals from sensors on the device or to external signals and controls the micropumps and cells. The implant device responds to biological signals and actively controls chemicals, biomolecules and cells to attract, retain, activate and deploy immune cells.

The implant device is a microelectrical mechanical system (MEMS) which incorporates micropumps, valves, reservoirs, conduits and chambers. For example a microfluidic device is fabricated from a silicon chip using photolithography and etching techniques to create reservoirs, conduits, chambers and electronic circuitry to control micropumps, electronic gates, and sensors. Microchip devices for drug delivery are described (see e.g., Stevenson et al., Advanced Drug Delivery Reviews 64: 1590-1602, 2012 and Razzacki et al., Advanced Drug Delivery Reviews 56: 185-198, 2004 which are incorporated herein by reference). Multiple reservoirs are constructed with associated micropumps and valves to control the delivery of chemicals, cells, or other immune cell stimuli to conduits and chambers in the device. For example a piezoelectric membrane pump with a flow rate of approximately 1 ml/min may be used to pump chemicals from the reservoirs (see e.g., Stevenson et al., Ibid.). The implant device is constructed with conduits and chambers arranged in pathways with reservoirs connected at various positions along the pathway. The immune cell implant device may contain one pathway that includes an entry port from the external tissues, a conduit leading to a chamber for immune cell accumulation and stimulation and an exit conduit leading to external tissues. Multiple reservoirs with associated micropumps and valves are connected to the chamber to provide cell attractants (e.g., chemokines), cell activators (e.g., agonists, cytokines, and antigens) and detachment agents (e.g., proteases, chelators). Microcircuitry on the implant device controls the micropumps to deliver chemicals and other immune cell stimuli from the reservoirs to the chambers and conduits. For example the micropump for a reservoir containing an immune cell attractant (e.g., chemokine) may be activated to pump a chemokine at a known concentration and flow rate for approximately 10-14 days into a chamber and the attached conduit which leads to external tissues. Then after approximately 10 days another micropump serving a second reservoir with a cell activator (e.g., cytokine) is activated to pump the cytokine into the same chamber at a predetermined flow rate for 1-2 days. Approximately 12-14 days after initiating chemokine pumping, a detachment agent (e.g. a protease) is pumped from a third reservoir to release adherent cells in the chamber and promote migration of immune cells to secondary lymphoid organs or to sites of infection or to cancer cells or to autoimmune cells. To promote emigration of the immune cells a fourth micropump serving a reservoir with buffered saline may be activated to pump at a predetermined flow rate once detachment is complete. The micropumps and valves of the implanted device may be programmed externally using wireless communication. The implanted device contains circuitry to receive signals from an external transmitter. Delivery of chemicals and other immune cell stimuli may be according to a predefined schedule and dose regimen which is preprogrammed from a computer terminal or dosing and scheduling may be done in real time by user signaling. For example the flow rate for delivery of a cytokine solution may be increased or decreased in response to blood tests or physiological parameters (e.g., body temperature) of the patient. Implanted microchips with wireless communication systems and computer interfaces which allow programming dose and schedule are described (see e.g., Farra et al., Science Translational Medicine 4, 122ra21 (2012 which is incorporated herein by reference).

The implanted device has sensors in the chambers and conduits to detect biochemicals, cells, or other immune cell stimuli. The sensors signal to the controller which in turn activates micropumps to increase or decrease pumping of attractants, antigens, cytokines, activators, or detachment reagents. Sensors to detect immune cell stimuli including cells are fabricated containing aptamer-based electrochemical sensors. Methods to select and produce aptamers (i.e., oligonucleotides with high affinity binding to molecular targets such as cell surface receptors or cytokines) are known (see e.g., U.S. Pat. No. 5,475,096, which is incorporated herein by reference). The construction of electrochemical sensors using microfabrication methods and employing aptamers to recognize specific biomolecules has been described (see e.g., U.S. Pat. No. 8,145,434; Lee et al., Anal. Bioanal. Chem. 390: 1023-1032, 2008 and U.S. Pat. No. 8,138,005; each of which are incorporated herein by reference). The biosensor may contain multiple electrodes coated with capture reagents, i.e., aptamers to form capacitive plates. Aptamers can be attached to the electrodes using a chemical linker, (e.g., succinic anhydride) which first bonds to the electrodes using amino-sialanization and then covalently couples with the aptamers. The apparatus is interfaced with electronic components which form a capacitance detector circuit. The detector circuit may include: an amplifier buffer, a current to voltage amplifier, resistors, and integration circuits. Binding of biomolecules to the immobilized aptamers changes the impedance at the electrode-solution interface, and changes in impedance are correlated with the amount of analyte bound to the immobilized aptamers (see e.g., U.S. Pat. No. 8,145,434, Ibid.). Each sensor may have multiple electrodes with different aptamers bound. The sensors are machined on silicon chips and include RFID tags to provide wireless communication and power harvesting to the sensors as well as to identify the sensors and indicate their location on the implant.

An RFID tag that includes antennas and circuitry to receive and transmit radio frequency signals that identify each sensor and the sensor's location on the implant is fabricated using microfabrication methods for MEMS. Methods and materials to construct RFID tags with antennas, transmitters, and power harvesters are described (see e.g., U.S. Pat. No. 7,479,886; and U.S. Pat. No. 7,411,505, each of which are incorporated herein by reference. The antenna may be a dipole antenna with a capacitor built in to store some of the electrical energy harvested from incident radio waves. The device may have a transmit circuit and a receive circuit to control radio wave communications through the antenna, a power harvester circuit to provide power to the device and a control circuit. The RFID tag may be constructed with circuitry to send an identification signal that includes location information, and to transmit an alert when biochemicals or cells are detected. Fabrication of RFID devices with integrated sensors as microchips has been described (see e.g., Sample et al., IEEE Trans. Instr. Meas. 57: 2608-2615, 2008 which is incorporated herein by reference).

Aptamers are used to retain immune cells in the chambers of the implant device. For example, aptamers with specificity for an immune cell surface molecule, (e.g., CD8), are attached to the surface of a chamber of the implant device. Methods to modify the surface of microfluidic chambers and to immobilize aptamers have been described (see e.g., Xu et al., Aptamer-Based Microfluidic Device for Enrichment, Sorting and Detection of Multiple Cancer Cells, Anal. Chem. 81: 7436-7442, 2009 which is incorporated herein by reference). To release aptamer-bound cells air is pumped into the chamber at approximately 200 nl/second and the cells are released as a bubble passes through the chamber.

### Prophetic Example 2

### An Implant Device is Programmed to Immunize a Patient Infected with Hepatitis C Virus

A patient infected with Hepatitis C virus (HCV) is immunized with a microfluidic implant device which actively controls immune cells and initiates an antiviral response. The device is implanted subcutaneously proximal to capillary beds which promote transport of cells and immune cell stimuli via the blood. The implant device has multiple reservoirs which contain a series of chemokines, cytokines, antigens, danger signals, and release agents to stimulate an anti-HCV immune response and deploy immune cells to sites of infection. Different pathways in the implant device are programmed to stimulate and deploy different immune cells with their corresponding effector functions. Immune cells and cytokines are recognized by sensors that signal to the controller on the device to apply a stimulus or release agent.

The implant device contains a pathway designed to initiate and sustain a cytotoxic T lymphocyte (CTL) response against HCV. The pathway includes an entry portal, a chamber and multiple reservoirs with valves and micropumps which deliver immune cell stimuli to the chamber. Biological agents associated with dendritic cell and CTL immune responses are described (see e.g., Satpathy et al., Nat. Immunol 13: 1145-1154, 2012 and Omar A. Ali, et al., Sci. Transl. Med. 1: 8ra19, 2009 which are incorporated herein by reference). For example an implant device may have multiple reservoirs as follows: Reservoir 1 contains Flt3 Ligand, a cytokine which promotes the generation of conventional dendritic cells (cDC) from hematopoetic precursors. Reservoir 2 contains granulocyte macrophage colony stimulating factor (GMCSF), an attractant for cDC, and cytosine-gaunosine oligodeoxynucleotide (CpG-ODN) which promotes the recruitment of CD8α⁺ DC, a dendritic cell subset efficient in presenting viral antigens to CTLs. Reservoir 3 contains HCV antigens, (e.g., HCV proteins). Reservoir 4 contains gamma interferon (γ-IFN) which promotes CTL development. Reservoir 5 contains air which is used to release immune cells from the chamber.

The anti-HCV CTL pathway in the device contains sensors to detect the presence of CD8α⁺ DC and CTL. Aptamer-based sensors (see Example 1) which recognize CD141 (a human CD8α⁺ DC marker), and CD8 (a marker for CTLs) are immobilized on the surface of the chamber to provide feedback when specific immune cell populations occupy the chamber. Signaling from the sensors is received by control circuitry on the device which starts or stops micropumps which control delivery of cytokines and other biological agents to the chamber. The time course and efficacy of antiviral immune responses display considerable variability between individuals and different viruses, for example influenza versus HCV. The time courses of antiviral immune responses and the corresponding programming of cytokines and immune stimulators are described (see e.g., Wherry and Ahmed, J. Virol. 78: 5535-5545, 2004 which is incorporated herein by reference). Responsive immunization with the implant device, based upon signaling from immune cell sensors, allows a personalized, pathogen specific immunization protocol. For example, a patient infected with HCV may be implanted with a device which is programmed to elicit an anti-HCV CTL response as follows:

| Reservoir/ | | |
|---|---|---|
| Biological | Start | Stop |
| Flt3 Ligand | Day 1 | Day 3 |
| GMCSF | Day 3 | Day 8 or CD8α⁺ DC |
| detected | | |
| CpG ODN | Day 3 | Day 8 orCD8α⁺ DC detected |
| HCV antigens | Day 3 | Day 8 or CD8α⁺ DC detected |
| γ-IFN | Day 10 or CTL detected | CTL detected |
| Release agent: | CTL detected | CTL undetected |
| e.g., air | | |

The start and stop of micropumps which control delivery of immune cell stimuli to the chamber may be programmed by computer (i.e., Day 1, Day 8, etc.) or it may be responsive to signals from the sensors in the chamber. For example if a sufficient number of CD8α⁺ DC is detected by the CD141 aptamer-based sensors then delivery of GMCSF, an attractant for DC, may be stopped. Alternatively, if clinical tests, (e.g., blood tests, physiological parameters) indicate the immunization program should be interrupted or changed the implant device may be programmed via wireless communication. For example, if adverse events attributed to γ-IFN are observed the delivery of γ-IFN may be stopped by “manual” intervention from a computer. If another round of immunization is required the implant device may be programmed to reinitiate the HCV CTL protocol.

The anti-HCV implant device may also contain a second type of pathway designed to elicit an antibody response targeting HCV. The pathway has a cell chamber with multiple reservoirs and micropumps providing immune cell stimuli to the chamber and to conduits leading in and out of the chamber. The immune cell types and biological modifiers associated with an IgG antibody response have been characterized. Conventional DC (cDC) present antigens and activate CD4⁺ helper T cells (T_{H}) which in turn activate B cells which respond to T_{H} and soluble antigen by the production of IgG antibodies. Moreover, follicular helper T cells (T_{FH}) interact with activated B cells to generate memory B cells and plasma cells which produce high affinity antibodies. Chemokines, cytokines and receptor ligands to attract and activate cDC, T_{H} cells, T_{FH} cells and B cells are described (see e.g., Gatto et al., Nat. Immunol. Published online: 17 Mar. 2013 doi:10.1038/ni.2555 which is incorporated herein by reference. A series of reservoirs may be designed to provide HCV antigens, and cell-specific immune cell stimuli at the time and place in the chamber optimal to elicit an anti-HCV IgG response. For example, GMCSF and 7α,25-dihydroxycholesterol (7α,25-OHC) are attractants for cDC and may be delivered simultaneously with HCV soluble protein antigens and a danger signal, e.g., CpG ODN, to activate antigen presentation. T_{H} cells may be attracted by the chemokine CCL13 and they are activated by cDC presenting HCV antigens and the delivery of IL-12 and γ-IFN. Then B cells may be attracted to the chamber by provision of the chemokines CCL19 and CCL21, and then activated by exposure to soluble HCV antigens and interaction with Th cells. IL-21 may be delivered to promote B cell activation and differentiation to memory B cells and plasma cells which produce large amounts of antibody.

The time course for humoral immune responses is established at approximately 14 days with timepoints at approximately 3 days to obtain activated B cells and 7 days to obtain plasma cells with IgG production at approximately 7-10 days. The implant device may be programmed to deliver the chemokines and cytokines and other immune cell stimuli on a predetermined schedule or in response to detection of the immune cell populations in the chamber. The implant may retain long lived plasma cells in the chamber using immobilized aptamers (see Example 1) and actively pump anti-HCV antibodies into the capillary beds surrounding the implant device or alternatively, allow the antibodies to diffuse to the surrounding tissues. Long lived plasma cells, which home to the bone marrow may be ejected from the implant device using air to disrupt the aptamer-bound cells (see Example 1 above).

At least a portion of the devices and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for detecting position and/or velocity, control motors for moving and/or adjusting components and/or quantities). A data processing system can be implemented utilizing suitable commercially available components, such as those typically found in data computing/communication and/or network computing/communication systems. In an embodiment, a feedback loop includes alerting the system by way of the computer circuitry that the device is empty, needs to be re-filled, or has intentionally or unintentionally shut down.

The herein described subject matter sometimes illustrates different components contained within, or connected with, different other components. It is to be understood that such depicted architectures are merely examples, and that in fact, many other architectures can be implemented that achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably coupleable,” to each other to achieve the desired functionality. Specific examples of operably coupleable include, but are not limited to, physically mateable and/or physically interacting components, and/or wirelessly interactable, and/or wirelessly interacting components, and/or logically interacting, and/or logically interactable components.

In an embodiment, one or more components may be referred to herein as “configured to,” “configurable to,” “operable/operative to,” “adapted/adaptable,” “able to,” “conformable/conformed to,” etc. Such terms (e.g., “configured to”) can generally encompass active-state components and/or inactive-state components and/or standby-state components, unless context requires otherwise.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by the reader that each function and/or operation within such block diagrams, flowcharts, or examples can be implemented, individually and/or collectively, by a wide range of hardware, software, firmware, or virtually any combination thereof. Further, the use of “Start,” “End,” or “Stop” blocks in the block diagrams is not intended to indicate a limitation on the beginning or end of any functions in the diagram. Such flowcharts or diagrams may be incorporated into other flowcharts or diagrams where additional functions are performed before or after the functions shown in the diagrams of this application. In an embodiment, several portions of the subject matter described herein is implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal-bearing medium used to actually carry out the distribution. Non-limiting examples of a signal-bearing medium include the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

While particular aspects of the present subject matter described herein have been shown and described, it will be apparent to the reader that, based upon the teachings herein, changes and modifications can be made without departing from the subject matter described herein and its broader aspects and, therefore, the appended claims are to encompass within their scope all such changes and modifications as are within the true spirit and scope of the subject matter described herein.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An implantable device, comprising:
a body structure having a surface including one or more actuatable reservoirs configured to direct an emitted biochemical or biological cell to one or more regions proximate the surface of the body structure and to deliver a patterned immune cell stimulus to the one or more regions proximate the surface of the body structure.

2. (canceled)

3. (canceled)

4. The implantable device of claim 1, further including one or more sensors.

5. The implantable device of claim 4, further including control circuitry is responsive to the one or more sensors.

6. The implantable device of claim 1, wherein the one or more sensors are configured to detect at least one biological cell or biochemical proximate the surface of the body structure; and
at least one computing device operably coupled to one or more of the plurality of spaced-apart independently actuatable reservoirs and configured to actuate one or more of the plurality of spaced-apart independently actuatable reservoirs between a reservoir discharge state and a reservoir retention state based on a comparison of a detected biological cell or biochemical to stored reference data.

7. The implantable device of claim 1, wherein the one or more sensors include at least one of an optical sensor, surface plasmon resonance sensor, biochemical sensor, MEMS sensor, magnetic sensor.

8. The implantable device of claim 1, further including:
control circuitry operably coupled to the one or more actuatable reservoirs and configured to control at least one of a spatial configuration parameter, or a temporal distribution parameter associated with the delivery of the patterned immune cell stimulus.

9. The implantable device of claim 1, further including:
a computing device operably coupled to the one or more actuatable reservoirs and configured to control at least one of a spatial distribution, or a temporal distribution associated with the delivery of the patterned immune cell stimulus.

10. The implantable device of claim 1, wherein the one or more actuatable reservoirs include a plurality of independently actuatable reservoirs.

11. The implantable device of claim 10, wherein one or more of the plurality of independently actuatable reservoirs are assigned to emit a first immune cell stimulus, and one or more of the plurality of independently actuatable reservoirs are assigned to emit a second immune cell stimulus.

12. (canceled)

13. (canceled)

14. (canceled)

15. (canceled)

16. (canceled)

17. (canceled)

18. The implantable device of claim 1, wherein at least one of the independent actuatable reservoirs is customized for a disease or condition of a specific biological subject.

19. (canceled)

20. (canceled)

21. (canceled)

22. (canceled)

23. (canceled)

24. The implantable device of claim 1, further including a detectable indicator including at least one of gold particles, magnetic particles, or a colorimetric dye.

25. (canceled)

26. (canceled)

27. (canceled)

28. (canceled)

29. (canceled)

30. The implantable device of claim 1, further including:
a plurality of spaced-apart independently actuatable reservoirs; and
at least one computing device operably coupled to one or more of the plurality of spaced-apart independently actuatable reservoirs and configured to actuate one or more of the plurality of spaced-apart independently actuatable reservoirs between a reservoir discharge state and a reservoir retention state.

31. The implantable device of claim 1, further including:
at least one receiver configured to acquire information based at least in part on whether a detected biological cell or biochemical proximate the surface of the body structure satisfies a target condition.

32. The implantable device of claim 1, further including at least one receiver operably coupled to control circuitry configured to one or more of the plurality of spaced-apart independently actuatable reservoirs and configured to actuate one or more of the plurality of spaced-apart independently actuatable reservoirs between a reservoir discharge state and a reservoir retention state based on a received signal.

33. The implantable device of claim 32, wherein the received signal is a wireless signal.

34. (canceled)

35. The implantable device of claim 32, wherein the received signal is a pre-programmed sequence command.

36. (canceled)

37. (canceled)

38. (canceled)

39. (canceled)

40. (canceled)

41. (canceled)

42. (canceled)

43. (canceled)

44. (canceled)

45. The implantable device of claim 31, further including one or more controllable microvalves configured to control access of one or more endogenous cells or endogenous biochemicals to the one or more regions proximate the surface of the body structure to an interior cavity of the body structure.

46. The implantable device of claim 45, wherein the interior cavity of the body structure is fluidically coupled to the one or more reservoirs.

47. The implantable device of claim 1, wherein the one or more reservoirs include at least one micropump or microvalve.

48. The implantable device of claim 45, further including at least one computing device operably coupled to the micropump or microvalve and configured to actuate the micropump or microvalve between a reservoir discharge state and a reservoir retention state based on a comparison of a detected biological cell or biochemical to stored reference data.

49. The implantable device of claim 1, further including at least one transmitter configured to send information based at least in part on a detected biological cell or biochemical.

50. (canceled)

51. The implantable device of claim 1, further including circuitry configured to obtain information and circuitry configured to store the obtained information.

52. The implantable device of claim 1, wherein the body structure includes at least one electroactive polymer, thermal-active polymer, magnetic-active polymer, or light-active polymer.

53. (canceled)

54. (canceled)

55. (canceled)

56. The implantable device of claim 1, wherein the immune cell stimulus is a vaccine.

57. (canceled)

58. (canceled)

59. (canceled)

60. (canceled)

61. (canceled)

62. (canceled)

63. The implantable device of claim 1, wherein the immune cell stimulus includes one or more cell regulatory molecules.

64. The implantable device of claim 63, wherein the one or more cell regulatory molecules include one or more of a cytokine, a chemokine, an antigen, a vaccine, an adjuvant, or a co-stimulatory molecule.

65. (canceled)

66. The implantable device of claim 1, further including at least one heating unit.

67. The implantable device of claim 66, wherein the at least one heating unit includes one or more of nickel, iron, or copper heating units.

68. The implantable device of claim 66, wherein the at least one heating unit includes a thermal conductive metal under application of an alternating magnetic field.

69. The implantable device of claim 66, wherein the at least one heating unit includes an inductive heating unit.

70. The implantable device of claim 66, wherein the at least one heating unit includes a wireless heating unit.

71. (canceled)

72. (canceled)

73. The implantable device of claim 1, further including at least one selectively activatable repelling mechanism.

74. (canceled)

75. (canceled)

76. (canceled)

77. (canceled)

78. The implantable device of claim 73, wherein the at least one selectively and activatable repelling mechanism includes one or more of a chemical, electrical, magnetic, electromagnetic, thermal, or other means.

79. The implantable device of claim 73, wherein the at least one selectively and activatable repelling mechanism includes one or more reservoirs containing at least one biochemical sufficient to repel an immune cell.

80. The implantable device of claim 73, wherein the at least one selectively and activatable repelling mechanism includes at least one electroactive polymer, thermal-active polymer, magnetic-active polymer, or light-active polymer.

81. The implantable device of claim 73, further including a power source.

82. (canceled)

83. The implantable device of claim 1, wherein at least one of the independently actuatable reservoirs are pre-loaded prior to implanting into a biological subject.

84. (canceled)

85. An implantable system, comprising:
a device having a body structure including
a surface with one or more sensors; and
a plurality of independently actuatable immune cell stimulus delivering reservoirs configured to deliver a patterned immune cell stimulus to the one or more regions proximate the surface of the body structure, the plurality of independently actuatable immune cell stimulus delivering reservoirs defining at least a portion of body structure.

86. A method of regulating an immune cell response from an at least partially implanted microfluidic device, comprising:
selectively and actively releasing one or more biochemicals or one or more biological cells to one or more regions proximate the surface of an implanted portion of the microfluidic device via one or more actuatable reservoirs, and
delivering a patterned immune cell stimulus composition to the one or more regions proximate the surface of the implanted portion of the microfluidic device in response to an automatically detected parameter associated with a biological sample proximate the surface of the implanted portion of the microfluidic device via one or more sensors of the device.

87. (canceled)

88. (canceled)

89. (canceled)

90. (canceled)

91. (canceled)

92. (canceled)

93. The method of claim 86, further including detecting at least one biological cell or at least one biochemical proximate the surface of the body structure of the implanted portion of the microfluidic device.

94. The method of claim 86, further including receiving information based at least in part on whether a detected biological cell or detected biochemical proximate the surface of the body structure that satisfies a target condition.

95. The method of claim 94, wherein the target condition includes at least one of a type of biological cell, type of biochemical, timing of delivery of an immune cell stimulus, response based on the type of surface of the body structure of the device, or spatial delivery of an immune cell stimulus.

96. The method of claim 86, further including sending information based at least in part on a detected biological cell or biochemical proximate the surface of the body structure that satisfies a target condition.
